(19) United States
(12) Patent Application Publication
(10) Pub. No.: US 2017/0275650 A1
(43) Pub. Date: Sep. 28, 2017

(54) ENDOSOMAL ESCAPE DOMAINS FOR DELIVERY OF MACROMOLECULES INTO CELLS

(71) Applicant: The Regents of the University of California, Oakland, CA (US)

(72) Inventors: Steven F. Dowdy, La Jolla, CA (US); Peter Lonn, Uppsala (SE)

(21) Appl. No.: 15/324,120

(22) PCT Filed: Jul. 22, 2015
(86) PCT No.: PCT/US15/41464

§ 371 Date: Jan. 5, 2017

Related U.S. Application Data
(60) Provisional application No. 62/027,513, filed on Jul. 22, 2014.

(51) Int. Cl. C12N 15/87; C07K 14/745; C07K 14/16; C07K 19/00; C07K 14/035; A61K 51/08; C07K 14/005; A61K 48/00

(52) U.S. Cl. CPC C12N 15/87; C07K 14/005; C07K 14/745; A61K 48/00; C07K 19/00; C07K 14/035; A61K 51/088; C07K 14/16; C12N 2720/12022; C07K 2319/00

(57) ABSTRACT

The disclosure provides fusion polypeptides and constructs useful for delivering diagnostics and therapeutics to cells. The fusion constructs include a protein transduction domain, a endosomal escape domain and a cargo domain. Also provided are methods of treating disease and disorders such as cell proliferative disorders.

### CROSS REFERENCE TO RELATED APPLICATIONS

The application claims priority under 35 U.S.C. §119 to U.S. Provisional Application Ser. No. 62/027,513, filed Jul. 22, 2014, the disclosure of which is incorporated herein by reference.

### STATEMENT REGARDING FEDERALLY SPONSORED RESEARCH

This invention was funded in part by Grant No. W81XWH-12-1-0141 awarded by the Department of Defense. The government has certain rights in the invention.

### TECHNICAL FIELD

This disclosure relates to fusion polypeptides comprising a transduction moiety and a therapeutic or diagnostic moiety.

### BACKGROUND

Eukaryotic cells contain several thousand proteins, which have been, during the course of evolution, selected to play specific roles in the maintenance of virtually all cellular functions. Not surprisingly, the viability of every cell, as well as the organism on the whole, is intimately dependent on the correct expression of these proteins. Factors which affect a particular protein's function, either by mutations or deletions in the amino acid sequence, or through changes in expression to cause over-expression or suppression of protein levels, invariably lead to alterations in normal cellular function. Such alterations often directly underlie a wide variety of genetic and acquired disorders. Consequently, the ability to target and selectively inhibit or kill cells comprising mutations that result in cell proliferative disorders would help to control such diseases and disorders.

In practice, however, the direct intracellular delivery of these agents has been difficult. This is due primarily to the bioavailability barrier of the plasma membrane, which effectively prevents the uptake of the majority of peptides and proteins and other agents by limiting their passive entry.

Traditionally, approaches to modulate protein function have largely relied on the serendipitous discovery of specific drugs and small molecules which could be delivered easily into the cell. However, the usefulness of these pharmacological agents is limited by their tissue distribution and unlike “information-rich” proteins, they often suffer from poor target specificity, unwanted side-effects, and toxicity. Likewise, the development of molecular techniques for gene delivery and expression of proteins has provided for advances in the understanding of cellular processes but has been of little benefit for the management of genetic disorders (Robbins et al., Trends Biotechnol. 16:35-40, 1998; Robbins and Ghivizzani, Pharmacol. Ther. 80:35-47, 1998).

### SUMMARY

The disclosure provides methods and compositions useful for delivery of molecules into cells. The compositions and methods generally comprise a protein transduction domain (PTD, sometimes referred to as a cell penetrating peptide (CPP)), and a plurality of aromatic ring structures spaced from the PTD domain.

The disclosure provides a fusion polypeptide comprising: (a) a protein transduction domain (PTD; sometimes referred to as a cell penetrating peptide (CPP)), the transduction domain comprising a membrane transport function; (b) an aromatic domain (e.g., a plurality of aromatic amino acids); and (c) a heterologous or cargo domain (e.g., a therapeutic and/or diagnostic agent), wherein the PTD is operably linked to the heterologous domain.

The disclosure provides a fusion polypeptide comprising (a) a protein transduction domain (PTD), the transduction domain comprising a membrane transport function; (b) an aromatic-rich peptide domain; and (c) a heterologous domain, wherein the PTD is operably linked to the aromatic-rich peptide domain and the heterologous domain. In one embodiment, the protein transduction domain is selected from the group consisting of a polypeptide comprising a herpesviral VP22 domain; a polypeptide comprising a human immunodeficiency virus (HIV) TAT domain; a polypeptide comprising a homeodomain of an Antennapedia protein (Antp HD) domain; an N-terminal cationic prion protein domain; and functional fragments thereof. In one embodiment, the protein transduction domain comprises a sequence selected from the group consisting of SEQ ID NO:7 from amino acid 47-57; B1-X₁-X₂-X₃-B₂-X₄-X₅-B₃, wherein B_{1,} B_{2,} and B₃ are each independently a basic amino acid, the same or different and X₁, X₂, X₃, X₄ and X₅ are each independently an alpha-helix enhancing amino acid the same or different (SEQ ID NO: 1) ; B₁-X₁-X₂-B₂-B₃-X₃-X₄-B₄, wherein B1, B2, B₃, and B₄ are each independently a basic amino acid, the same or different and X₁, X₂, X₃, and X₄ are each independently an alpha-helix enhancing amino acid the same or different (SEQ ID NO:2); X-X-R-X-(P/X)-(B/X)-B-(P/X)-X-B-(B/X), wherein X is any alpha helical promoting residue such as alanine; P/X is either proline or X as previously defined, B is a basic amino acid residue and B/X is either B or X as defined above (SEQ ID NO:4); a sequence of about 7 to 10 amino acids and containing KX₁RX₂X₁, wherein X₁ is R or K and X₂ is any amino acid (SEQ ID NO:5); RKKRRQRRR (SEQ ID NO:6); and KKRPKPG (SEQ ID NO:3). In another embodiment the heterologous domain comprises a diagnostic and/or therapeutic agent. In a further embodiment, the therapeutic agent is a thrombolytic agent or an anticellular agent. In still a further embodiment, the thrombolytic agent comprises streptokinase or urokinase. In one embodiment, the therapeutic agent is an anticellular agent. In one embodiment, the anticellular agent is selected from the group consisting of a chemotherapeutic agent and a mammalian cell cytotoxin. In a further embodiment, the chemotherapeutic agent is selected from the group consisting a steroid, an antimetabolite, an anthracycline, a vinca alkaloid, an antibiotic, an alkylating agent, an epipodophyllotoxin, neocarzinostatin (NCS), adriamycin and dideoxycytidine. In another embodiment, the mammalian cell cytotoxin is selected from the group consisting of interferon-α (IFN-α), interferon-βγ (IFN-βγ), interleukin-12 (IL-12) and tumor necrosis factor-α (TNF-α). In one embodiment, the anticellular agent is selected from the group consisting of plant-, fungus- and bacteria-derived toxins. In a further embodiment, the toxin is selected from the group consisting of a ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain. In yet another embodiment, the therapeutic agent comprises a radioactive moiety comprising a radioisotope. In one embodiment, the therapeutic agent is an anti-cancer agent. In a further embodiment, the anti-cancer agent inhibits cell proliferation. In still a further embodiment, the anti-cancer agent is a suicide gene or a tumor suppressor protein. In yet still a further embodiment, the suicide gene is thymidine kinase or cytosine deaminase. In another embodiment, the tumor suppressor protein is p53. In one embodiment, the diagnostic agent is selected from the group consisting of a fluorgenic agent, a paramagnetic agent and a radioactive agent. In a further embodiment, the paramagnetic agent comprises an ion selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III) ions. In yet a further embodiment, the radioactive agent comprises an ion selected from the group consisting of iodine¹²³, technicium^{99m}, indium ¹¹¹, rhenium¹⁸⁸, rhenium^{186,} copper⁶⁷, iodine¹³¹, yttrium⁹⁰, iodine¹²⁵, astatine²¹¹, gallium⁶⁷, iridium¹⁹², cobalt⁶⁰, radium²²⁶, gold¹⁹⁸, cesium¹³⁷ and phosphorus³² ions. In yet an alternative embodiment, the fluorogenic agents is selected from the group consisting of gadolinium and renographin. In one embodiment, the aromatic-rich peptide domain comprises 1 to 8 amino acids and comprises from 3-5 aromatic rings. In any of the foregoing embodiments, the aromatic rich peptide domain further comprises a hydrophilic polymer spacer between the PTD and the aromatic-rich peptide domain. In a further embodiment, the hydrophilic polymer spacer comprises polyethylene glycol having 1-18 (e.g., 1, 2, 3, 4, 5, 6, 7, 8, 9, 10, 11, 12, 13, 14, 15, 16, 17 or 18) PEG moieties. In a further embodiment of any of the foregoing embodiments, the aromatic-rich peptide domain is an endosomal escape domain (EED) comprising the aromatic-rich peptide domain and a PEG linker. In a further embodiment, the endosomal escape domain (EED) comprises of 1 to 8 amino acids comprising from 3-5 aromatic groups and a spacer of 2-18 PEG moieties. In yet another embodiment, the EED comprises 4 aromatic groups. In a further embodiment, the EED does not comprise more than 3 phenylalanines in series (e.g., no more than 2 adjacent phenylalanines). In one embodiment, the aromatic-rich peptide domain comprises a peptide selected from the group consisting of GFFG, GWG, GFWG, GFWFG, GWWG and GWGGWG. In one embodiment, the fusion polypeptide has the general formula: Z-PTD-(PEG)ₓ-(aromatic amino acids)₂₋₄ wherein x is 2-18 and Z is the heterologous domain. In a further embodiment, the fusion polypeptide has the general formula selected from the group consisting of: Z-PTD-(PEG)ₓ-GFFG, Z-PTD-(PEG)ₓ-GWG, Z-PTD-(PEG)ₓ-GFWG, Z-PTD-(PEG)ₓ-GFWFG, Z-PTD-(PEG)ₓ-GWWG and Z-PTD-(PEG)ₓ-GWGGWG.

The disclosure also provides a pharmaceutical composition comprising the fusion polypeptide of any of the foregoing embodiments.

The disclosure also provides a method of introducing a therapeutic and/or diagnostic agent in to a target cell, the method comprising contacting the cell with the fusion polypeptide or pharmaceutical composition of the disclosure. The contacting can be in vitro or in vivo.

The disclosure also provides a method of treating a cell proliferative disorder in a subject, comprising contacting the subject with a fusion polypeptide or a pharmaceutical composition of the disclosure. In one embodiment, the fusion polypeptide further comprises a ligand domain comprising a ligand that binds to a cell surface marker expressed on a cell comprising a cell proliferative disorder. In yet a further embodiment, the ligand domain comprises DV3.

The disclosure also provides a method of identifying a cell comprising a phenotype of interest in a subject, the method comprising contacting the subject with a fusion polypeptide of the disclosure, wherein the heterologous domain comprises a diagnostic agent.

The disclosure also provides a fusion polypeptide comprising (a) a protein transduction domain (PTD), the transduction domain comprising a membrane transport function; and (b) a peptide comprising SEQ ID NO:28. In one embodiment, the protein transduction domain is selected from the group consisting of a polypeptide comprising a herpesviral VP22 domain; a polypeptide comprising a human immunodeficiency virus (HIV) TAT domain; a polypeptide comprising a homeodomain of an Antennapedia protein (Antp HD) domain; an N-terminal cationic prion protein domain; and functional fragments thereof. In a further embodiment, the protein transduction domain comprises a sequence selected from the group consisting of SEQ ID NO:7 from amino acid 47-57; B1-X₁-X₂-X₃-B₂-X₄-X₅-B₃, wherein B₁, B₂, and B₃ are each independently a basic amino acid, the same or different and X₁, X₂, X₃, X₄ and X₅ are each independently an alpha-helix enhancing amino acid the same or different (SEQ ID NO:1); B₁-X₁-X₂-B₂-B₃-X₃-X₄-B_{4,} wherein B₁, B_{2,} B₃, and B₄ are each independently a basic amino acid, the same or different and X₁, X₂, X₃, and X₄ are each independently an alpha-helix enhancing amino acid the same or different (SEQ ID NO:2); X-X-R-X-(P/X)-(B/X)-B-(P/X)-X-B-(B/X), wherein X is any alpha helical promoting residue such as alanine; P/X is either proline or X as previously defined, B is a basic amino acid residue and B/X is either B or X as defined above (SEQ ID NO:4); a sequence of about 7 to 10 amino acids and containing KX₁RX₂X₁, wherein X₁ is R or K and X₂ is any amino acid (SEQ ID NO:5); RKKRRQRRR (SEQ ID NO:6); and KKRPKPG (SEQ ID NO:3). In another embodiment, the fusion polypeptide further comprises (i) a heterologous domain comprises a diagnostic and/or therapeutic agent, (b) an endosomal escape domain, (c) a targeting ligand domain or (d) any combination thereof.

The disclosure also provides a method of measuring transport of a molecule into a cell comprising contacting a cell comprising the N-terminal domain of green fluorescent protein comprising a sequence that is at least 90% identical to SEQ ID NO:27 from amino acid 1-214, with a fusion polypeptide comprising a peptide having a sequence of SEQ ID NO:28 and measuring fluorescence.

The disclosure also provides an isolated polynucleotide encoding any of the fusion polypeptides described herein. In yet a further embodiment, the disclosure provides a vector comprising the polynucleotide. The disclosure also provides a host cell containing the vector.

The disclosure also provides an assay system comprising a simple real-time, quantitative live cell phenotypic PTD/CPP transduction assay using a split GFP peptide cargo complementation approach that allows for a direct measurement of the transduced cargo in the cytoplasm.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-G shows transduction of GFPβ11-TAT induces fluorescence complementation of intracellularly expressed GFPβ1-10 protein fragment. (A) Shows the study concept. PTD/CPP binding to the cell surface stimulates macropinocytotic uptake and endosomal escape of GFPβ11-PTD/CPP peptide into the cytoplasm. Binding of GFPβ11 peptide to non-fluorescent GFPβ1-10 protein fragment in the cytoplasm induces chemical formation of the GFP fluorescent chromophore. (B) Shows a dose-dependent comparison of GFPβ1-10 expressing H1299c#G3 human lung adenocarcinoma cells treated with GFPβ11-TAT peptide, or control GFPβ11 peptide plus TAT peptide (in trans) analyzed by FACS. The graph shows mean values of triplicate samples with S.D. (C) Is a histograms of GFPβ1-10 expressing H1299c#G3 human lung adenocarcinoma cells treated with increasing doses of GFPβ11-TAT peptide, or control GFPβ11 peptide plus TAT peptide (in trans) analyzed by FACS. (D, E) Shows cell viability (D) and morphology (FSC/SSC) (E) of GFPβ1-10 H1299c#G3 cells treated with increasing doses of GFPβ11-TAT peptide or control GFPβ11 peptide plus TAT peptide. The graphs show mean values of triplicate samples with S.D. (F) Shows a kinetic analysis of GFPβ1-10 H1299c#G3 cells treated with 40 μM GFPβ11-TAT peptide and control GFPβ11 peptide plus TAT peptide. The graph shows mean values of triplicate samples with S.D. (G) Is a histogram of GFPβ1-10 H1299c#G3 cells treated with GFPβ11-TAT peptide and control untreated and measured by FACS.

FIG. 2A-F shows GFPβ11-TAT transduction efficiency in multiple cell-types. (A-C) Shows a FACS analysis of GFPβ1-10 expressing HaCatc#G7 (A), MCF7c#G7 (B), and MDA-MB-231c#G3 (C) cells treated with 60 μM GFPβ11-TAT peptide or not. Bar graphs represent mean values of triplicate samples with S.D. (D-F) Show histograms of HaCatc#G7 (D), MCF7c#G7 (E), and MDA-MB-231c#G3 (F) cells treated with 60 μM GFPβ11-TAT peptide or untreated control and measured by FACS.

FIG. 3A-C shows Comparison of PTD/CPP delivery domains. (A) Dose-dependent comparison of GFPβ1-10 expressing H1299c#G3 cells treated with GFPβ11-TAT or GFPβ11-(S-S)-TAT. The graph displays single sample measurements. (B) GFPβ1-10 expressing H1299c#G3 cells were treated with 30 μM of GFPβ11-(S-S)-PTD/CPP peptides, as indicated, and analyzed by FACS. The bar graph displays mean values of triplicate samples with S.D. GFPβ11-(S-S)-TP10 could not be determined due to high toxicity (N.D.) (C) GFPβ1-10 expressing H1299c#G3 cells were treated with 30 μM of the indicated GFPβ11-PTDs/CPPs −/+ 100 μM Chloroquine. GFP fluorescence was determined by FACS analysis and mean fluorescence was graphed as fold increase over non-treated control cells. The bar graphs show mean values of triplicate samples with S.D.

FIG. 4A-D shows optimizing endosomal escape by introducing PEG-spacers between PTD/CPP delivery domain and a hydrophobic patch. (A-D) Dose-dependent comparison of GFPβ1-10 expressing H1299c#G3 cells treated with GFPβ11-(S-S)-TAT-PEG(n)-GWWG (SEQ ID NOs:31 and 33-35) (A) peptides containing varying length (n) of PEG spacer (P) analyzed for GFP fluorescence (B), cellular morphology (C), and number of viable cells (D) by FACS. The graphs show mean values of triplicate sample analysis with S.D.

FIG. 5A-G shows optimizing design of endosomal escape domain (EED). (A-D) Dose-dependent comparison of GFPβ1-10 H1299-c#G3 cells treated with GFPβ11-(S-S)-TAT-(X) peptides containing a PEG6-spaced aromatic ring hydrophobic endosomal escape domain (EED), as indicated (SEQ ID NOs:32, 33, 36-41), to parental GFPβ11-(S-S)-TAT peptide and control GFPβ11-(S-S)-TAT-PEG6-GG peptide analyzed by FACS for GFP fluorescence (A, B), cellular morphology (C) and cell viability (D). The table (A) displays mean values from triplicate samples and the graphs (B, D, D) show the same mean values with S.D. error bars (E-G) Dose-dependent comparison of GFPβ1-10 expressing H1299c#G3 cells treated with GFPβ11-(S-S)-TAT-(EED) peptides containing four aromatic ring hydrophobic residues PEG6-GFWFG (SEQ ID NO:39), PEG6-GWWG (SEQ ID NO:33), or PEG6-GFFFFG (SEQ ID NO:42), to parental GFPβ11-(S-S)-TAT peptide by FACS for GFP fluorescence (E), cellular morphology (F) and cell viability (G). The graphs display mean values of triplicate samples with S.D.

FIG. 6A-L shows an evaluation of GFPβ11-(S-S)-TAT-PEG6-GFWFG peptide in multiple cell types. (A-D) Dose-dependent analysis of GFPβ1-10 expressing HaCaTc#G7 keratinocytes treated with GFPβ11-(S-S)-TAT-PEG6-GFWFG peptide by FACS for GFP fluorescence (A, B), cellular morphology (C) and cell viability (D). (E-H) Dose-dependent analysis of GFPβ1-10 expressing MDA-MB-231c#G3 breast carcinoma cells treated with GFPβ11-(S-S)-TAT-PEG6-GFWFG peptide by FACS for GFP fluorescence (E, F), cellular morphology (G) and cell viability (H). (I-L) Dose-dependent analysis of GFPβ1-10 expressing MCF7c#G7 breast carcinoma cells treated with GFPβ11-(S-S)-TAT-PEG6-GFWFG peptide by FACS for GFP fluorescence (I, J), cellular morphology (K) and cell viability (L). Graphs display mean values of triplicate samples with S.D.

FIG. 7 is a diagram depicting aspects of the disclosure. Positively charged TAT-PTD/CPP binds to yet unknown receptors/molecules at the cell membrane in the extracellular space that in turn leads to Rac1 activation, actin reorganization and macropinocytosis. Inside the endosomal lumen, the optimize EED motif buries itself into the membrane to enhance endosomal escape without damaging the cell membrane.

FIG. 8A-D shows the characterization of GFPβ1-10 expressing H1299c#G3/c#G4 human lung adenocarcinoma cells. (A) Western blot of expression levels of GFPβ1-10 in H1299, H1299c#G3, and H1299c#G4 cells. Blotting with anti-tubulin was used as loading control. (B-D) Dose-dependent comparison of GFPβ11-TAT treated H1299, H1299c#G3, and H1299c#G4 human lung adenocarcinoma cells treated with peptide, and control GFPβ11 peptide plus control TAT peptide (in trans) analyzed by FACS. Graphs display mean values of triplicate samples with S.D.

FIG. 9A-B shows maturation of recombinant GFPβ11-TAT/GFP-β1-10 at 37° C. (A, B) GFPβ11-TAT was incubated together with GFPβ1-10 in PBS for different time-points at 37° C. before being imaged on an IVIS-imager (A) and plotted as a graph of fold GFP fluorescence over background (B). The graph displays single sample measurements. Duplicate samples were compared (I, II).

FIG. 10A-B shows that inhibiting macropinocytosis blocks GFPβ11-TAT cellular uptake. (A) GFPβ1-10 expressing H1299c#G3 cells were transfected with siCTRL or siRAC1 before being treated with 20 μM or 40 μM GFPβ11-TAT or 40 μM GFPβ11 peptide as control. Samples were measured for GFP fluorescence on FACS. The bar graphs show mean values of triplicate samples with S.D. (B) GFPβ1-10 expressing MDA-MB-231c#G3 were pretreated with or without 80 μM EIPA before being transduced with 60 μM GFPβ11-TAT with or without 80 μM EIPA for 40 min or 80 min and assayed for GFP fluorescence on FACS. DMSO was used as vehicle control. The bar graphs show mean values of triplicate samples with S.D.

### DETAILED DESCRIPTION

As used herein and in the appended claims, the singular forms “a,” “and,” and “the” include plural referents unless the context clearly dictates otherwise. Thus, for example, reference to “a PTD” includes a plurality of such PTDs and reference to “the linker” includes reference to one or more linkers, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

The ability to deliver functional agents to cells is problematical due to the bioavailability restriction imposed by the cell membrane. That is, the plasma membrane of the cell forms an effective barrier, which restricts the intracellular uptake of molecules to those which are sufficiently non-polar and smaller than approximately 500 daltons in size. Previous efforts to enhance the internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway. In addition, liposomal formulations can be cytotoxic.

An advantage of protein transduction is the intracellular delivery of proteins or agents which are otherwise difficult to transfect and where microinjection is not a possible option. For instance, primary lymphocytes are very difficult to transfect, requiring electroporation of DNA constructs. This process is very inefficient, killing 90-99% of the cells, and yielding protein expression in less than 10% of those which survive.

PTDs/CPPs have been used to deliver therapeutic cargo into cells in culture, studied in pre-clinical models of disease and are currently in clinical trials. There are over 100 published PTD/CPP delivery domain sequences; however, most published PTDs/CPPs have only been investigated using dye-labeled molecules. Consequently, excluding cell death, there is a paucity of quantitative transduction assays that rely on robust and well-controlled controlled cellular phenotypes that can be readily quantified to determine which PTDs/CPPs are the most efficient and least cytotoxic delivery domains. Previous attempts to develop phenotypic based transduction assays have either relied on signal amplification steps (e.g. splice-correction, Cre recombination), or membrane permeable cargo that when non-specifically cleaved from the PTD/CPP may result in a high false-positive rate (e.g. Luciferin), or cargo that induced cell death, which is a difficult phenotype to separate cargo delivery effects from PTD/CPP induced cytotoxicity.

The disclosure provides an assay system and compositions useful for promoting and studying protein transduction using PTDs/CPPs. The disclosure provides a number of embodiments, using a PTD/CPP molecule linked to a cargo molecule and comprising a domain that promotes fusogenic activity.

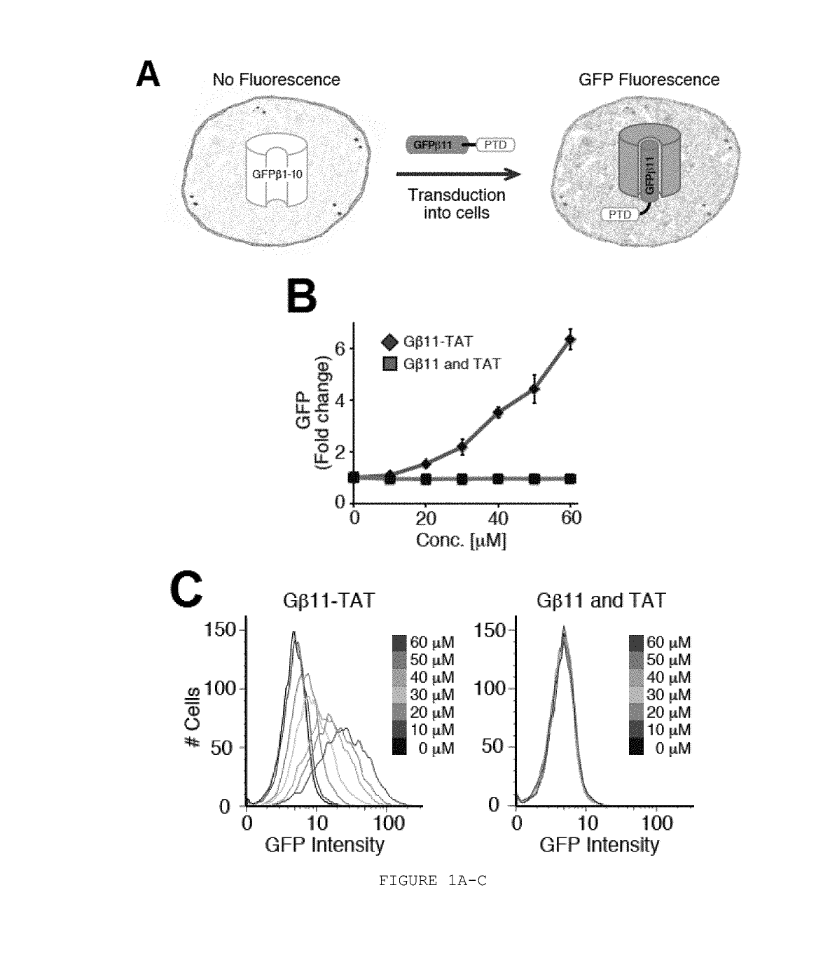

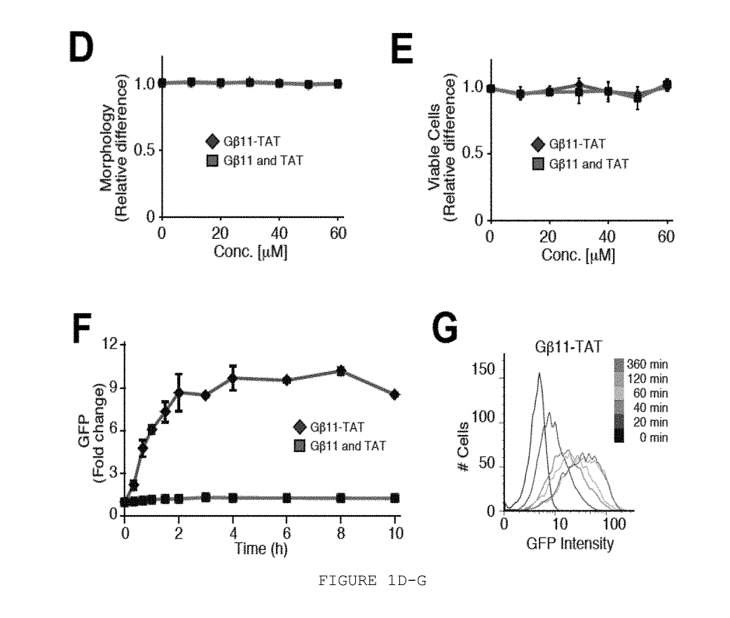

The disclosure describes and provides, in one embodiment, a simple real-time, quantitative live cell phenotypic PTD/CPP transduction assay using a split GFP peptide cargo complementation approach that for the first time allows for a direct measurement of the transduced cargo in the cytoplasm. The split GFP fluorescent complementation assay provided by the disclosure, uses live cells, allows for concurrent measurement of morphology and cytotoxicity, and has a near zero false-positive rate due the fact that the 16 residue GFPβ11 peptide is too large (1,826 Da) to enter cells on its own (membrane impenetrable) and that in the absence of the GFPβ11 peptide, the GFPβ1-10 protein fragment has no ability to fluoresce on its own (no chromophore formation possible) (FIG. 1a). The small synthetic size and low cost of the 16 amino acid GFPβ11 peptide cargo combined with FACS instruments present in most academic departments or core facilities, allows the ability to readily assay protein transduction.

Briefly, PTD/CPP delivery of macromolecules into the cytoplasm requires: 1) cellular association and uptake by endocytosis, and 2) escape from the endosome into the cytoplasm, which is the rate-limiting delivery step. Using the GFPβ11 fluorescence complementation assay a study was performed to analyze PTD/CPP activity. In this regard, a head-to-head comparison of different PTDs/CPPs was performed. The data show that Arginine containing PTDs/CPPs rapidly transduce macromolecular cargo into cells with the highest efficiency, showing measureable activity above background as soon as 20 min post-addition and reaching a cytoplasmic maximum by 2 h post-addition. In contrast, low Arginine abundance and predominantly hydrophobic PTDs/CPPs although effective to transduce cargo into a cell were poor transducers and/or cytotoxic to be efficiently used as delivery agents. Arginine residues contain a bi-dentate guanidinium cationic charge that forms an ionic bond with cell surface bi-dentate anionic counterpart sulfates, phosphates and carboxylic acid groups present on sugars, lipids and proteins. These cationic charges stimulate macropinocytosis uptake and facilitating endosomal activity. In contrast, Lysine's mono-dentate cationic charge failed to stimulate uptake or endosomal escape.

As mentioned above, even with effective uptake, escape from endosomes remains the rate-limiting step for delivery of macromolecular cargo into the cytoplasm by all delivery agents, including PTDs/CPPs and LNPs. It is estimated that only a small fraction of the endosomal bound (cell associated) TAT-PTD/CPP escapes from the macropinosome into the cytoplasm, perhaps as little as or even less than 1%. Consistent with this notion, addition of chloroquine, an endolytic proton sponge, resulted in a ˜4 fold increase of GFPβ11-TAT fluorescence complementation of GFPβ1-10. Using a systematic approach, and the assay systems described herein, the disclosure provides endosomal escape domains having compositions that improve escape from the endosome of PTD transported cargo. For example, the disclosure demonstrates that an Endosomal Escape Domain (EED) composition of 3-5 aromatic ring containing residues (e.g., four aromatic ring containing hydrophobic or polar residues such as FWF or WW) were useful. The term “residue” includes both naturally occurring amino acids and unnatural amino acids.

Thus, not only does the disclosure provide an assay system, but the disclosure also provides fusion polypeptides for delivery and endosomal escape. The disclosure provides chimeric/fusion polypeptides comprising a PTD and a heterologous molecule (i.e., a cargo molecule). In one embodiment, the chimeric/fusion polypeptide comprises a PTD linked to a heterologous molecule such as a polynucleotide, a small molecule, or a heterologous polypeptide domain and comprising a domain having 3-5 aromatic rings that promote endosomal escape. In one embodiment, the chimeric/fusion polypeptide comprises a PTD linked to the heterologous polypeptide and a hydrophobic domain or a peptide domain with 3-5 aromatic groups.

The disclosure provides fusion polypeptides and compositions useful in cellular transduction and cellular modulation. The fusion polypeptides of the disclosure comprise a transduction moiety domain comprising a membrane transport function and a heterologous domain (e.g., a therapeutic or diagnostic agent) linked to or separated by a hydrophobic domain or a peptide domain with 3-5 aromatic groups. In some embodiments, additional domains including, but not limited to, targeting domains and the like can be linked to the fusion polypeptides of the disclosure. For example, a multi-domain approach can be used to selectively target fusion polypeptides comprising a PTD domain to a desired cell type. In one embodiment, the multi-domain approach can be used to selectively target anticancer agents to a tumor cell and thereby selectively kill tumor cells based on receptor overexpression, common to many malignancies. Due to the inherent absence of a size limitation on transduction domains to deliver therapeutic cargo into cells, the disclosure can be applied reiteratively to refine both the tumor selectivity and killing abilities of multi-domain transducible macromolecules to further enhance therapeutic efficacy.

A number of protein transduction domains/peptides are known in the art and have been demonstrated to facilitate uptake of heterologous molecules (e.g., cargo molecules) linked to the transduction domain. Such transduction domains facilitate uptake through a process referred to a macropinocytosis. However, macropinocytosis is a nonselective form of endocytosis that all cells perform.

The discovery of several proteins which can efficiently pass through the plasma membrane of eukaryotic cells has led to the identification of a novel class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O′Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins (the foregoing references are all incorporated herein by reference). Not only can these proteins/polypeptides pass through the plasma membrane but the attachment of other molecules, such as the enzyme β-galactosidase, was sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type and can efficiently transduce 100% of cells in culture with no apparent toxicity (Nagahara et al., Nat. Med. 4:1449-52, 1998). In addition to full-length proteins, protein transduction domains have also been used successfully to induce the intracellular uptake of DNA (Abu-Amer, supra), antisense oligonucleotides (Astriab-Fisher et al., Pharm. Res, 19:744-54, 2002), small molecules (Polyakov et al., Bioconjug. Chem. 11:762-71, 2000) and even inorganic 40 nanometer iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is no apparent size restriction to this process.

The fusion of a protein transduction domain (PTD) with a heterologous molecule (e.g., a polynucleotide, small molecule, or protein) is sufficient to cause their transduction into a variety of different cells in a concentration-dependent manner. Moreover, this technique for protein delivery appears to circumvent many problems associated with DNA and drug based techniques.

PTDs/CPPs are typically cationic in nature. These cationic protein transduction domains track into lipid raft endosomes carrying with them their linked cargo and release their cargo into the cytoplasm by disruption of the endosomal vesicle. Examples of PTDs include AntHD, TAT, VP22, cationic prion protein domains and functional fragments thereof. The disclosure provides methods and compositions that combine the use of PTDs such as TAT and poly-Arg with a heterologous (e.g., “cargo”) domain. The two domains (e.g., the PTD and heterologous/cargo domain) are linked to one another by one or more linkers. Furthermore, the PTD-cargo fusion polypeptide can include additional domains including fusogenic peptides and/or targeting peptides (e.g., ligands for cell surface receptors or cognates). These compositions provide methods whereby a therapeutic or diagnostic agent can be taken up by the process of micropinocytosis.

In general, the transduction domain of the fusion molecule can be nearly any synthetic or naturally-occurring amino acid sequence that can transduce or assist in the transduction of the fusion molecule. As mentioned previously, the PTDs/CPPs TAT, VP22, and AntHD are well studied and their structure and sequences have been characterized and manipulated. For example, transduction can be achieved in accord with the disclosure by use of a protein sequence such as an HIV TAT protein or fragment thereof that is covalently linked at the N-terminal or C-terminal end to the heterologous/cargo domain. Alternatively, the transducing protein can be the Antennapedia homeodomain or the HSV VP22 sequence, the N-terminal fragment of a prion protein or suitable transducing fragments thereof such as those known in the art. In other embodiments, the transduction domain can be a synthesized sequence that comprises characteristics of TAT, VP22 and/or AntHD.

The type and size of the PTD will be guided by several parameters including the extent of transduction desired. PTDs will be capable of transducing at least about 20%, 25%, 50%, 75%, 80% or 90% of the cells of interest, more preferably at least about 95%, 98% and up to, and including, about 100% of the cells. Transduction efficiency, typically expressed as the percentage of transduced cells, can be determined by several conventional methods.

PTDs will manifest cell entry and exit rates (sometimes referred to as k₁ and k₂, respectively) that favor at least picomolar amounts of the fusion molecule in the cell. The entry and exit rates of the PTD and any cargo can be readily determined or at least approximated by standard kinetic analysis using detectably-labeled fusion molecules or the assay as described more fully herein below. Typically, the ratio of the entry rate to the exit rate will be in the range of between about 5 to about 100 up to about 1000.

In one embodiment, a PTD useful in the methods and compositions of the disclosure comprise a peptide featuring substantial alpha-helicity. It has been discovered that transduction is optimized when the PTD exhibits significant alpha-helicity. In another embodiment, the PTD comprises a sequence containing basic amino acid residues that are substantially aligned along at least one face of the peptide. A PTD domain of the disclosure may be a naturally occurring peptide or a synthetic peptide.

In another embodiment of the disclosure, the PTD comprises an amino acid sequences comprising a strong alpha helical structure with arginine (Arg) residues down the helical cylinder.

In yet another embodiment, the PTD domain comprises a peptide represented by the following general formula: B1-X₁-X₂-X₃-B₂-X₄-X₅-B₃ (SEQ ID NO:1) wherein B₁, B₂, and B₃ are each independently a basic amino acid, the same or different; and X₁, X₂, X₃, X₄ and X₅ are each independently an alpha-helix enhancing amino acid the same or different.

In another embodiment, the PTD domain is represented by the following general formula: B₁-X₁-X₂-B₂-B₃-X₃-X₄-B₄ (SEQ ID NO:2) wherein B₁, B₂, B₃, and B₄ are each independently a basic amino acid, the same or different; and X₁, X₂, X₃, and X₄ are each independently an alpha-helix enhancing amino acid the same or different.

Additionally PTD domains comprise basic residues, e.g., lysine (Lys) or arginine (Arg), and further including at least one proline (Pro) residue sufficient to introduce “kinks” into the domain. Examples of such domains include the transduction domains of prions. For example, such a peptide comprises KKRPKPG (SEQ ID NO:3).

In one embodiment, the domain is a peptide represented by the following sequence: X-X-R-X-(P/X)-(B/X)-B-(P/X)-X-B-(B/X) (SEQ ID NO:4), wherein X is any alpha helical promoting residue such as alanine; P/X is either proline or X as previously defined; B is a basic amino acid residue, e.g., arginine (Arg) or lysine (Lys); R is arginine (Arg) and B/X is either B or X as defined above.

In another embodiment the PTD is cationic and consists of between 7 and 10 amino acids and has the formula KX₁RX₂X₁ (SEQ ID NO:5) wherein X₁ is R or K and X₂ is any amino acid. An example of such a peptide comprises RKKRRQRRR (SEQ ID NO:6).

Additional transducing domains in accordance with this disclosure include a TAT fragment that comprises at least amino acids 49 to 56 of TAT up to about the full-length TAT sequence (see, e.g., SEQ ID NO:7). A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment the impede alpha helix formation or stability. In a more specific embodiment, the TAT fragment will include at least one amino acid substitution with an alpha-helix enhancing amino acid. Typically the TAT fragment will be made by standard peptide synthesis techniques although recombinant DNA approaches may be used in some cases.

Additional transduction proteins (PTDs) that can be used in the compositions and methods of the disclosure include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence. In a more specific embodiment, the substitution is chosen so that at least two basic amino acid residues in the TAT 49-56 sequence are substantially aligned along at least one face of that sequence.

Also included are chimeric PTD domains. Such chimeric transducing proteins include parts of at least two different transducing proteins. For example, chimeric transducing proteins can be formed by fusing two different TAT fragments, e.g., one from HIV-1 and the other from HIV-2 or one from a prion protein and one from HIV etc.

The ability of PTDs to transduce heterologous (i.e., cargo) domains into cells have been successfully demonstrated in vitro and in vivo. Examples of PTDs fused with various heterologous domains is provided in Table 1. These applications cover a broad range of uses and, in general, there appears to be no particular limitation in either the size or type of protein that can be delivered. TAT protein transduction has been useful in a variety of situations to overcome the limitations of traditional DNA-based approaches or for the development of novel strategies in the treatment of disease.

TABLE 1

| TAT-Protein | Effect | References |
|---|---|---|
| TAT-Bcl-xL | anti-apoptotic | Cao et al., (2002) J. |
| | | Neurosci. 22, 5423-31, |
| | | Kilic et al., (2002) Ann. |
| | | Neurol. 52, 617-22, |
| | | Dietz et al., (2002) Mol. |
| | | Cell Neurosci. 21, 29-37, |
| | | Embury et al., (2001) |
| | | Diabetes 50, 1706-13 |
| TAT-p53 | tumor suppressor | Takenobu et al., (2002) |
| | protein | Mol. Cancer Ther. 1, |
| | | 1043-9 |
| TAT-ARC | transduction into | Gustafsson et al., (2002) |
| | myocardium is | Circulation 106, 735-9 |
| | cardioprotective | |
| TAT-cyclin | E restoration of | Hsia et al., (2002) Int. |
| | proliferation | Immunol. 14, 905-16 |
| TAT-glutamate | restoration of | Yoon et al., (2002) |
| dehydrogenase | GDH-deficiency | Neurochem. Int. 41, 37- |
| | disorders | 42 |
| TAT-Cu, Zn-SOD | antioxidant protein | Kwon et al., (2000) |
| | | FEBS Lett. 485, 163-7, |
| | | Eum et al., (2002) Mol. |
| | | Cells 13, 334-40 |
| TAT-catalase | antioxidant protein | Jin et al., (2001) Free |
| | | Radic. Biol. Med. 31, |
| | | 1509-19 |
| TAT-ODD- | anti-tumor activity | Harada et al., 2002) |
| Caspase 3 | | Cancer Res. 62, 2013-8 |
| TAT-HIV1- | specific killing of | Vocero-Akbani et al., |
| Caspase 3 | HIV-infected cells | (1999) Nat. Med. 5, 29- |
| | | 33 |
| TAT-Cre | site-specific | Joshi et al., (2002) |
| | recombination | Genesis. 33, 48-54, Peitz |
| | | et al., (2002) Proc. Natl. |
| | | Acad. Sci. USA 99, |
| | | 4489-94 |
| TAT-APOBEC | editing of ApoB mRNA | Yang et al., (2002) Mol. |
| | | Pharmacol. 61, 269-76 |
| TAT-GFP | fluorescent protein | Caron et al., (2001) Mol. |
| | | Ther. 3, 310-8, Han et |
| | | al., (2001) Mol. Cells 12, |
| | | 267-71 |
| TAT-H-Ras | cytoskeletal | Hall et al., (2001) Blood |
| | reorganization | 98, 2014-21 |
| TAT-IkappaB | NF-kappaB | Abu-Amer et al., 2001) |
| | inhibitory protein | J. Biol. Chem. 276, |
| | | 30499-503. |
| TAT-HPC-1/ | inhibitor of | Fujiwara et al., (2001) |
| syntaxin | neurotransmitter | Biochim. Biophys. Acta |
| | release | 1539, 225-32 |
| TAT-p16 | inhibitor of cyclin | Ezhevsky et al., (2001) |
| | D/cdk complexes | Mol. Cell Biol. 21, 4773- |
| | | 84 |
| TAT-p27 | cyclin-dependent | McAllister et al., (2003) |
| | kinase inhibitor | Mol. Cell Biol. 23, 216- |
| | | 28 |
| TAT-b- | frequently used | Barka et al., (2000) J. |
| galactosidase | reporter enzyme | Histochem. Cytochem. |
| | | 48, 1453-1460, |
| | | Schwarze et al., (1999) |
| | | Science 285, 1569-72 |
| TAT-p21 | cell cycle arrest | Kunieda et al., (2002) |
| | in G1 phase | Cell Transplant 11, 421- |
| | | 8 |
| TAT-PEA-15 | prevents apoptosis | Embury et al., (2001) |
| | by TNFa in pancreatic | Diabetes 50, 1706-13 |
| | cell line | |
| TAT-beta- | lysosomal enzyme | Xia et al., (2001) Nat. |
| glucuronidase | | Biotechnol. 19, 640-4 |

A PTD/CPP as described herein are linked to a heterologous or cargo domain to form a fusion polypeptide. In one embodiment, the fusion polypeptide comprises a PTD as described above (e.g., SEQ ID NO:1-6 or 7) operably linked to a polypeptide comprising SEQ ID NO:27 from amino acid 214-238 (e.g., a GFPβ11 fragment) or SEQ ID NO:28. In a further embodiment, the fusion polypeptide comprises a domain comprising an endosomal escape domain (EDD) linked to the PTD, the GFPβ11 domain or separating the PTD and GFPβ11 domain. In another embodiment, a PTD/CPP as described herein are linked to a heterologous or cargo domain to form a fusion polypeptide. The heterologous/cargo domain can be any polypeptide, small molecule, nucleic acid etc. to be delivered. In this embodiment, the fusion polypeptide comprises a domain comprising an endosomal escape domain (EDD) linked to the PTD, the heterologous/cargo domain or separating the PTD and the heterologous/cargo domain. The endosomal escape domain comprises a domain of aromatic residues (e.g., natural or unnatural amino acids) comprising 3-5 aromatic moieties.

The term “operably linked” or “operably associated” refers to functional linkage between two domains (e.g., a PTD and EED, cargo domain etc. or in the case of polynucleotides, a regulatory sequence and the polynucleotide regulated by the regulatory sequence as well as the link between encoded domains of the fusion polypeptides such that each domain is linked in-frame to give rise to the desired polypeptide sequence).

By the term “fusion polypeptide” as it is used herein is meant a transducing molecule such as a PTD protein or peptide sequence covalently linked (e.g., fused) to one or more heterologous polypeptides (e.g., a polypeptide, small molecule etc. and, in some embodiments, an endosomal escape domain and may further include additional domains) by recombinant, chemical or other suitable method. If desired, the fusion polypeptide can be fused at one or several sites through a peptide linker. The peptide linker can comprise one or more sites for cleavage by a pathogen induced- or host cell induced-protease. Alternatively, the peptide linker may be used to assist in construction of the fusion polypeptide or to assist in purification of the fusion polypeptide.

As used herein and endosomal escape domain (EED) refers to a domain of 1-8 amino acids comprising from 2-6 aromatic groups (e.g., tryptophan has 2 aromatic groups) and a spacer of from 2-18 polyethylene glycol (PEG) moieties. In one embodiment, the EED comprises 4 aromatic groups. In a further embodiment, the EED does not comprise more than 3 phenylalanines in series. In another embodiment, the EED comprises amino acids having aromatic rings that are spaced from one another by at least one non-aromatic containing amino acids. In another embodiment, the EED comprises a peptide selected from the group consisting of GFFG, GWG, GFWG, GFWFG, GWWG and GWGGWG or unnatural amino acids having structure that correspond to G, W, or F. In another embodiment of any of the foregoing, the EED comprises from 1-18 PEG moieties. In a further embodiment, the EED comprise 3-8 PEG moieties. In a specific embodiment, the EED comprises 6 PEG moieties.

The disclosure thus provides a protein transduction domain linked to an EED domain. For example, the PTD-EDD can have the general structure: PTD-(PEG)ₓ-(aromatic amino acids)₂₋₄ wherein x is 1-18. In one embodiment the aromatic amino acids can be flanked by a non-aromatic amino acid or may include non-aromatic amino acids separating one or more aromatic amino acids. For example, the PTD-EED can have a structure selected from the group consisting of: PTD-(PEG)ₓ-GFFG, PTD-(PEG)ₓ-GWG, PTD-(PEG)ₓ-GFWG, PTD-(PEG)ₓ-GFWFG, PTD-(PEG)ₓ-GWWG and PTD-(PEG)ₓ-GWGGWG, wherein x is 1-18. The PTD may be linked to additional domains, such as a cargo domain, targeting domain or fusogenic peptide domain. In such embodiments, a fusion polypeptide of the disclosure can have the general structure: Z-PTD-(PEG)ₓ-(aromatic amino acids)₂₋₄, wherein x is 1-18 and wherein Z is a cargo domain or heterologous polypeptide.

A transducible cargo-PTD-EED (e.g., cargo-PTD-(PEG)ₓ-(aromatic amino acids)₂₋₄) enhances release of cargo or heterologous molecules from the endosome into the cytoplasm, nucleus or other cellular organelle.

Peptide linkers that can be used in the fusion polypeptides and methods of the disclosure will typically comprise up to about 20 or 30 amino acids, commonly about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the heterologous/cargo domain and/or other domains. For example, the peptide linker sequence can be positioned between the protein transduction domain and the heterologous domain, e.g., to provide molecular flexibility. The length of the linker moiety is chosen to optimize the biological activity of the polypeptide comprising a PTD domain-ligand domain fusion and a heterologous molecule and can be determined empirically without undue experimentation. The linker moiety should be long enough and flexible enough to allow a ligand of the fusion construct to freely interact with its binding partner. Examples of linker moieties are -Gly-Gly-, -S-S-, GGGGS (SEQ ID NO:9), (GGGGS)_{N} (SEQ ID NO:10), GKSSGSGSESKS (SEQ ID NO:11), GSTSGSGKSSEGKG (SEQ ID NO:12), GSTSGSGKSSEGSGSTKG (SEQ ID NO:13), GSTSGSGKPGSGEGSTKG (SEQ ID NO:14), or EGKSSGSGSESKEF (SEQ ID NO:15). Linking moieties are described, for example, in Huston et al., Proc. Nat'l Acad. Sci 85:5879, 1988; Whitlow et al., Protein Engineering 6:989, 1993; and Newton et al., Biochemistry 35:545, 1996. Other suitable peptide linkers are those described in U.S. Pat. Nos. 4,751,180 and 4,935,233, which are hereby incorporated by reference.

The methods, compositions, and fusion polypeptides of the disclosure provide enhanced uptake and release of PTDs linked to heterologous molecules. A PTD fusion polypeptide can comprise a PTD domain, an EED domain, and a heterologous domain with or without additional domains (e.g., fusogenic domains, receptor or ligand domains, polyethylene glycol domains and the like).

PTDs can be linked or fused with any number of ligand domains, directly or indirectly. The ligand domains serve one or more purposes including, for example, to target the fusion polypeptide to a target cell expressing the ligand's cognate receptor and/or to promote uptake of the fusion polypeptide. Furthermore, the fusion polypeptide comprising the PTD and the ligand domain can be linked to any number of heterologous molecules having, for example, a therapeutic and/or diagnostic effect.

A ligand domain (e.g., a targeting molecule) for use in the invention includes, but is not limited to, a ligand or an antibody that specifically binds to its corresponding target, for example, a receptor on a cell surface. Thus, for example, where the ligand domain is an antibody, the fusion polypeptide will specifically bind (target) cells and tissues bearing the epitope to which the antibody is directed. Thus, a ligand refers generally to all molecules capable of reacting with or otherwise recognizing or binding to a receptor or polypeptide on a target cell. Any known ligand or targeting molecule can be used as the ligand domain of the fusion polypeptide of the disclosure. Examples of targeting peptides that can be manipulated and cloned or linked to produce a fusion polypeptide are ample in the literature. In general, any peptide ligand can be used or fragments thereof based on the receptor-binding sequence of the ligand. In immunology, such a peptide domain is referred to as an epitope, and the term epitope may be used herein to refer to a ligand recognized by a receptor. For example, a ligand comprises the sequence of a protein or peptide that is recognized by a binding partner on the surface of a target cell, which for the sake of convenience is termed a receptor. However, it should be understood that for purposes of the invention, the term “receptor” encompasses signal-transducing receptors (e.g., receptors for hormones, steroids, cytokines, insulin, and other growth factors), recognition molecules (e.g., MHC molecules, B- or T-cell receptors), nutrient uptake receptors (such as transferrin receptor), lectins, ion channels, adhesion molecules, extracellular matrix binding proteins, and the like that are located and accessible at the surface of the target cell.

A number of chemokine ligands are known in the art. For example, DV3 is used in the Examples herein; however other chemokine ligands are known in the art (see, e.g., Zhou et al., J. Biol. Chem., 277(20):17476-17485, 2002, incorporated herein by reference).

The size of the ligand domain peptide can vary within certain parameters. Examples of ligands include, but are not limited to, antibodies, lymphokines, cytokines, receptor proteins such as CD4 and CD8, hormones, growth factors, and the like which specifically bind desired target cells. For example, several human malignancies overexpress specific receptors, including HER2, LHRH and CXCR4. Accordingly, ligands to these receptors can be used in the fusion polypeptides, methods and compositions of the invention. Receptor ligand domains are known in the art.

As used herein, a “fusogenic” domain is any polypeptide that facilitates the destabilization of a cell membrane or the membrane of a cell organelle. For example, the hemagglutinin (HA) of influenza is the major glycoprotein component of the viral envelope. It has a dual function in mediating attachment of the virus to the target cell and fusion of the viral envelope membrane with target cell membranes. In the normal course of viral infection, virus bound to the cell surface is taken up into endosomes and exposed to relatively low pH. The pH change triggers fusion between the viral envelope and the endosomal membrane, as well as conformational changes in HA, which lead to increased exposure of the amino terminus. Synthetic peptides such as the N-terminus region of the influenza hemagglutinin protein destabilize membranes. Examples of HA2 analogs include GLFGAIAGFIEGGWTGMIDG (SEQ ID NO:15) and GLFEAIAEFIEGGWEGLIEG (SEQ ID NO:16).

Other fusogenic proteins include, for example, the M2 protein of influenza A viruses employed on its own or in combination with the hemagglutinin of influenza virus or with mutants of neuraminidase of influenza A, which lack enzyme activity, but which bring about hemagglutination; peptide analogs of the influenza virus hemagglutinin; the HEF protein of the influenza C virus, the fusion activity of the HEF protein is activated by cleavage of the HEFo into the subunits HEF1 and HEF2; the transmembrane glycoprotein of filoviruses, such as, for example, the Marburg virus, the Ebola virus; the transmembrane glycoprotein of the rabies virus; the transmembrane glycoprotein (G) of the vesicular stomatitis virus; the fusion polypeptide of the Sendai virus, in particular the amino-terminal 33 amino acids of the F1 component; the transmembrane glycoprotein of the Semliki forest virus, in particular the E1 component, the transmembrane glycoprotein of the tickborn encephalitis virus; the fusion polypeptide of the human respiratory syncytial virus (RSV) (in particular the gp37 component); the fusion polypeptide (S protein) of the hepatitis B virus; the fusion polypeptide of the measles virus; the fusion polypeptide of the Newcastle disease virus; the fusion polypeptide of the visna virus; the fusion polypeptide of murine leukemia virus (in particular p15E); the fusion polypeptide of the HTL virus (in particular gp21); and the fusion polypeptide of the simian immunodeficiency virus (SIV). Viral fusogenic proteins are obtained either by dissolving the coat proteins of a virus concentration with the aid of detergents (such as, for example, β-D-octylglucopyranoside) and separation by centrifugation (review in Mannio et al., BioTechniques 6, 682 (1988)) or else with the aid of molecular biology methods known to the person skilled in the art.

The heterologous domain (i.e., cargo domain) of the fusion polypeptide of the disclosure can comprise a therapeutic agent and/or a diagnostic agent. Examples of selected agents include therapeutic agents, such as thrombolytic agents and anticellular agents that kill or suppress the growth or cell division of disease-associated cells (e.g., cells comprising a cell proliferative disorder such as a neoplasm or cancer). Examples of effective thrombolytic agents are streptokinase and urokinase.

Effective anticellular agents include classical chemotherapeutic agents, such as steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α(IFN-α), interferon-βγ (IFN-βγ), interleukin-12 (IL-12) and tumor necrosis factor-a (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

Diagnostic agents will generally be a fluorogenic, paramagnetic or radioactive ion that is detectable upon imaging. Examples of paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III) ions.

Examples of radioactive ions include iodine¹²³, technicium^{99m}, indium¹¹¹, rhenium¹⁸⁸, rhenium¹⁸⁶, copper⁶⁷, iodine¹³¹, yttrium⁹⁰, iodine^{125,} astatine²¹¹, gallium⁶⁷, iridium¹⁹², cobalt⁶⁰, radium²²⁶, gold¹⁹⁸, cesium¹³⁷ and phosphorus³² ions. Examples of fluorogenic agents include gadolinium and renographin.

In attaching a fluorogenic, paramagnetic or radioactive ion to a fusion polypeptide of the disclosure, the agent is linked to the protein or polypeptide carrier, using methods commonly known in the art.

As used herein, a heterologous domain can be (1) any heterologous polypeptide, or fragment thereof, (2) any polynucleotide (e.g., a ribozyme, antisense molecule, polynucleotide, oligonucleotide and the like); (3) any small molecule, or (4) any diagnostic or therapeutic agent, that is capable of being linked or fused to protein backbone (e.g., linked or fused to a PTD or ligand domain). For example, PTD fusion molecule can comprise a PTD-ligand domain linked to a heterologous polypeptide, or fragment thereof that provides a therapeutic effect when present in a targeted cell.

The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; anti-inflammatory agents (e.g., soluble TNF receptor domains such as ENBREL); tumor suppressor proteins; clotting factors including Factor VIII and Factor IX, protein S, protein C, antithrombin III, von Willebrand Factor, cystic fibrosis transmembrane conductance regulator (CFTR), and negative selective markers such as Herpes Simplex Virus thymidine kinase.

In addition, a heterologous molecule fused to the PTD-EED domain can be a negative selective marker or “suicide” protein, such as, for example, the Herpes Simplex Virus thymidine kinase (TK). Such a PTD linked to a suicide protein may be administered to a subject whereby tumor cells are selectively transduced. After the tumor cells are transduced with the kinase, an interaction agent, such as gancyclovir or acyclovir, is administered to the subject, whereby the transduced tumor cells are killed. Growth of the tumor cells is inhibited, suppressed, or destroyed upon expression of the anti-tumor agent by the transduced tumor cells.

In addition, a heterologous molecule can be a diagnostic agent such as an imaging agent. For example, a PTD-EED fusion polypeptide can be fused to a radio-labeled moiety.

Thus, it is to be understood that the disclosure is not to be limited to any particular heterologous domain used for diagnosis and/or treatment of any particular disease or disorder. Rather, the heterologous domain can be any domain known or used in other fusion proteins in the art for treatment or delivery of diagnostic or therapeutic agents.

The polypeptides used in the disclosure (e.g., with respect to particular domains of a fusion polypeptide or the full length fusion polypeptide) can comprise either the L-optical isomer or the D-optical isomer of amino acids or a combination of both. Polypeptides that can be used in the disclosure include modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. For example, a functional fragment of a PTD includes a fragment which retains transduction activity. Biologically functional fragments, for example, can vary in size from a polypeptide fragment as small as an epitope capable of binding an antibody molecule, to a large polypeptide capable of participating in the characteristic induction or programming of phenotypic changes within a cell. An “epitope” is a region of a polypeptide capable of binding an immunoglobulin generated in response to contact with an antigen. Small epitopes of receptor ligands can be useful in the methods of the invention so long as it retains the ability to interact with the receptor.

In some embodiments, retro-inverso peptides are used. “Retro-inverso” means an amino-carboxy inversion as well as enantiomeric change in one or more amino acids (i.e., levantory (L) to dextrorotary (D)). A polypeptide of the disclosure encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995).

As noted, components of the fusion polypeptides disclosed herein, e.g., a PTD domain, an EED domain, a heterologous domain, and optionally peptide linkers, can be organized in nearly any fashion provided that the fusion polypeptide has the function for which it was intended. The disclosure provides fusion polypeptides or chimeric proteins comprising one or more PTDs linked either directly or indirectly to a heterologous domain (e.g., a therapeutic or diagnostic agent) and includes an EED domain linked to either the PTD or heterologous domain. In some embodiments, the fusion polypeptide may including additional domains (e.g., targeting domains, polyethylene glycol spacers and the like). Each of the several domains may be directly linked or may be separated by a linker peptide. The domains may be presented in any order (e.g., PTD-heterologous domain-EDD; EDD-PTD-heterologous domain; EDD-heterologous domain-PTD; heterologous domain-PTD-EDD; and similar variations). Additionally, the fusion polypeptides may include tags, e.g., to facilitate identification and/or purification of the fusion polypeptide, such as a 6xHIS tag.

In another embodiment, the disclosure provides a method of producing a fusion polypeptide comprising a PTD domain, an aromatic peptide domain and a heterologous molecule (and optionally additional domains) by growing a host cell comprising a polynucleotide encoding the fusion polypeptide under conditions that allow expression of the polynucleotide, and recovering the fusion polypeptide. A polynucleotide encoding a fusion polypeptide of the disclosure can be operably linked to a promoter for expression in a prokaryotic or eukaryotic expression system. For example, such a polynucleotide can be incorporated in an expression vector.

Accordingly, the disclosure also provides polynucleotides encoding a fusion protein construct of the disclosure. Such polynucleotides comprise sequences encoding a PTD domain, an aromatic peptide sequence of 2-6 amino acids, and a heterologous domain operably linked in any order. The polynucleotide may also encode linker domains that separate one or more of the PTD, aromatic domain and heterologous domains.

A polynucleotide of the disclosure can be introduced into a cell using a variety of methods known to those of skill in the art. For example, a construct comprising such a polynucleotide can be delivered into a cell using a colloidal dispersion system. Alternatively, a polynucleotide construct can be incorporated (i.e., cloned) into an appropriate vector. For purposes of expression, the polynucleotide encoding a fusion polypeptide of the disclosure may be inserted into a recombinant expression vector. The term “recombinant expression vector” refers to a plasmid, virus, or other vehicle known in the art that has been manipulated by insertion or incorporation of a polynucleotide encoding a fusion polypeptide of the disclosure. The expression vector typically contains an origin of replication, a promoter, as well as specific genes that allow phenotypic selection of the transformed cells. Vectors suitable for such use include, but are not limited to, the T7-based expression vector for expression in bacteria (Rosenberg et al., Gene, 56:125, 1987), the pMSXND expression vector for expression in mammalian cells (Lee and Nathans, J. Biol. Chem., 263:3521, 1988), baculovirus-derived vectors for expression in insect cells, cauliflower mosaic virus, CaMV, and tobacco mosaic virus, TMV, for expression in plants.

Depending on the vector utilized, any of a number of suitable transcription and translation elements (regulatory sequences), including constitutive and inducible promoters, transcription enhancer elements, transcription terminators, and the like may be used in the expression vector (see, e.g., Bitter et al., Methods in Enzymology, 153:516-544, 1987). These elements are well known to one of skill in the art.

In yeast, a number of vectors containing constitutive or inducible promoters may be used (see, e.g., Current Protocols in Molecular Biology, Vol. 2, Ed. Ausubel et al., Greene Publish. Assoc. & Wiley Interscience, Ch. 13, 1988; Grant et al., “Expression and Secretion Vectors for Yeast,” in Methods in Enzymology, Eds. Wu & Grossman, Acad. Press, N.Y., Vol. 153, pp.516-544, 1987; Glover, DNA Cloning, Vol. II, IRL Press, Wash., D.C., Ch. 3, 1986; “Bitter, Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982). A constitutive yeast promoter, such as ADH or LEU2, or an inducible promoter, such as GAL, may be used (“Cloning in Yeast,” Ch. 3, R. Rothstein In: DNA Cloning Vol.11, A Practical Approach, Ed. DM Glover, IRL Press, Wash., D.C., 1986). Alternatively, vectors may be used which promote integration of foreign DNA sequences into the yeast chromosome.

An expression vector can be used to transform a host cell. By “transformation” is meant a permanent genetic change induced in a cell following incorporation of a polynucleotide exogenous to the cell. Where the cell is a mammalian cell, a permanent genetic change is generally achieved by introduction of the polynucleotide into the genome of the cell. By “transformed cell” or “recombinant host cell” is meant a cell into which (or into an ancestor of which) has been introduced, by means of molecular biology techniques, a polynucleotide encoding a fusion polypeptide of the invention. Transformation of a host cell may be carried out by conventional techniques as are known to those skilled in the art. Where the host is prokaryotic, such as E. coli, competent cells which are capable of polynucleotide uptake can be prepared from cells harvested after exponential growth phase and subsequently treated by the CaCl₂ method by procedures known in the art. Alternatively, MgCl₂ or RbCl can be used. Transformation can also be performed after forming a protoplast of the host cell or by electroporation.

A fusion polypeptide of the disclosure can be produced by expression of polynucleotide encoding a fusion polypeptide in prokaryotes. These include, but are not limited to, microorganisms, such as bacteria transformed with recombinant bacteriophage DNA, plasmid DNA, or cosmid DNA expression vectors encoding a fusion polypeptide of the disclosure. The constructs can be expressed in E. coli in large scale. Purification from bacteria is simplified when the sequences include tags for one-step purification by nickel-chelate chromatography. Thus, a polynucleotide encoding a fusion polypeptide can also comprise a tag to simplify isolation of the fusion polypeptide. For example, a polyhistidine tag of, e.g., six histidine residues, can be incorporated at the amino terminal end of the fusion polypeptide. The polyhistidine tag allows convenient isolation of the protein in a single step by nickel-chelate chromatography. A fusion polypeptide of the disclosure can also be engineered to contain a cleavage site to aid in protein recovery the cleavage site may be part of a linker moiety as discussed above. A DNA sequence encoding a desired peptide linker can be inserted between, and in the same reading frame as, a polynucleotide encoding a heterologous domain, a PTD, or fragment thereof followed by a plurality of aromatic amino acids, using any suitable conventional technique. For example, a chemically synthesized oligonucleotide encoding the linker can be ligated between two coding polynucleotides. In particular embodiments, a polynucleotide of the disclosure will encode a fusion polypeptide comprising from three to four separate domains (e.g., a PTD domain, an aromatic peptide domain and a heterologous polypeptide domain) are separated by peptide linkers.

When the host cell is a eukaryotic cell, such methods of transfection of DNA as calcium phosphate co-precipitates, conventional mechanical procedures, such as microinjection, electroporation, insertion of a plasmid encased in liposomes, or virus vectors may be used. Eukaryotic cells can also be cotransfected with a polynucleotide encoding the PTD-fusion polypeptide of the disclosure, and a second polynucleotide molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the fusion polypeptide (see, e.g., Eukaryotic Viral Vectors, Cold Spring Harbor Laboratory, Gluzman ed., 1982).

Eukaryotic systems, and typically mammalian expression systems, allow for proper post-translational modifications of expressed mammalian proteins to occur. Eukaryotic cells that possess the cellular machinery for proper processing of the primary transcript, glycosylation, phosphorylation, and advantageously secretion of the fusion product can be used as host cells for the expression of the PTD-fusion polypeptide of the disclosure. Such host cell lines may include, but are not limited to, CHO, VERO, BHK, HeLa, COS, MDCK, Jurkat, HEK-293, and WI38.

For long-term, high-yield production of recombinant proteins, stable expression is used. Rather than using expression vectors that contain viral origins of replication, host cells can be transformed with the cDNA encoding a fusion polypeptide of the disclosure controlled by appropriate expression control elements (e.g., promoter, enhancer, sequences, transcription terminators, polyadenylation sites, and the like), and a selectable marker. The selectable marker in the recombinant plasmid confers selectivity (e.g., by cytotoxin resistance) and allows cells to stably integrate the plasmid into their chromosomes and grow to form foci that, in turn, can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy et al., Cell, 22:817, 1980) genes can be employed in tk-, hgprt- or aprt-cells, respectively. Also, antimetabolite resistance can be used as the basis of selection for dhfr, which confers resistance to methotrexate (Wigler et al., Proc. Natl. Acad. Sci. USA, 77:3567, 1980; O′Hare et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin et al., J. Mol. Biol., 150:1, 1981); and hygro, which confers resistance to hygromycin genes (Santerre et al., Gene, 30:147, 1984). Additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase), which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

Techniques for the isolation and purification of either microbially or eukaryotically expressed PTD-fusion polypeptides of the disclosure may be by any conventional means, such as, for example, preparative chromatographic separations and immunological separations, such as those involving the use of monoclonal or polyclonal antibodies or antigen.

The fusion polypeptides of the disclosure are useful for the treatment and/or diagnosis of a number of diseases and disorders. For example, the fusion polypeptides can be used in the treatment of cell proliferative disorders, wherein the fusion polypeptide (e.g., heterologous domain-PTD-EDD) and wherein the heterologous domain comprises a cytotoxic agent are delivered to a cancer cell. The PTD domain facilitates uptake of the fusion polypeptide and the EED domain facilitates release of the cargo from the endosome. Thus, the fusion polypeptide is useful for treatment and, when comprising a ligand domain, can selective target cells having cell proliferative disorders. Similarly, the fusion polypeptides of the disclosure can be used to treatment inflammatory diseases and disorders, infections, vascular disease and disorders and the like.

Typically a fusion polypeptide of the disclosure will be formulated with a pharmaceutically acceptable carrier, although the fusion polypeptide may be administered alone, as a pharmaceutical composition.

A pharmaceutical composition according to the disclosure can be prepared to include a fusion polypeptide of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a fusion polypeptide according to the disclosure necessary to prevent, to cure, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g, subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The working examples below are provided to illustrate, not limit, the invention. Various parameters of the scientific methods employed in these examples are described in detail below and provide guidance for practicing the invention in general.

### EXAMPLES

Plasmids, antibodies, siRNAs and other reagents. Mammalian optimized pCMV-mGFP-β1-10plasmid (22004005) was purchased from Sandia Biotech. EIPA, Chloroquine, and DMSO was from Sigma. Anti-GFP (Invitrogen) and anti-α-Tubulin (Sigma) were used for immunoblotting. siRNA targeting human RAC1 (ID: s11711) and control siRNA (4611G) was bought from Ambion. Lipofectamine 2000 was purchased from Invitrogen.

Cell culture, transfections and immunoblots. H1299, MCF7, MDA-MB-231 and HaCat cells were maintained in DMEM supplemented with 10% FBS, 100 U/ml penicillin, and 100 U/ml streptomycin. H1299c#G3, H1299c#G4, MCF7c#G7, MDA-MB-231c#G3 and HaCaTc#G7 cells were generated by transfecting cells with pCMV-mGFPβ1-10 and subsequently grown under hygromycin selection. Hygromycin resistant cells were then treated with GFPβ11-TAT and directly after, individual, transiently fluorescent clonal cells were isolated by FACS sorting. Clones were expanded and tested for stable GFPβ1-10 expression. H1299c#G3, H1299c#G4, MCF7c#G7, MDA-MB-231c#G3 and HaCaTc#G7 cells were maintained in DMEM supplemented with 10% FBS, 100 U/ml penicillin, 100 U/ml streptomycin and 80 or 100 ug/ml hygromycin. Transient transfections of siRNA or plasmid DNA were performed using Lipofectamine 2000 according to standard protocol. Immunblots were performed using 10% SDS-PAGE, semi-dry transfer (BioRad) and developed on ChemiDoc Imager (BioRad).

Synthesis of peptides. Fmoc solid phase peptide synthesis was performed using a Symphony Quartet peptide synthesizer (Ranin) and rink-amide MBHA resin as solid support. Protected amino acids and coupling reagents were purchased from Anaspec. Synthesized peptides were cleaved and deprotected using standard conditions (95% TFA with water and TIS) and subsequently precipitated using cold diethylether. Prep-scale RP-HPLC with an Agilent Prep C18 30×250 mm column was used to for purification and peptide purity and size was confirmed by mass spectrometry using a-CHCA matrix (Voyager, Applied Biosystems DE-Pro MALDI-TOF). Peptides were then lyophilized and resuspended in pure water or in pure water with 5% glycerol and stored at −20° C. for short term or at −80° C. for long term.

Disulfide conjugation. GFP-β11-Cystein was combined with NPYS protected Cystein-PTD/CPP at 1:1.5 or 1.5:1 ratio. pH was adjusted to -7.5 using PBS or Tris-HCl. Reactions were incubated 1 h at RT before being purified using HPLC. Conjugation and purity of products was confirmed by mass spectrometry using α-CHCA matrix (Voyager, Applied Biosystems DE-Pro MALDI-TOF). Peptides were then lyophilized and resuspended in pure water with 5% glycerol and stored at −20° C. for short term or at −80° C. for long term.

In vitro complex formation of GFP-β¹¹-TAT and GFP-β¹⁻¹⁰. GFP-β11-TAT was incubated with recombinant GFP-β1-10 for the indicated time-points in PBS on a black opaque 96-well plate at 37° C. The plate was analyzed for GFP fluorescence using an IVIS Spectrum imager.

Peptide transduction. All transduction experiments were performed in 48-well plates. An optimized protocol was established. First, 15,000 or 20,000 cells were plated in each well. Next day, the indicated peptides were pipetted into eppendorf tubes. Transduction buffer (60% OptiMEM and 40% PBS) was added to peptide (100 μl total volume), directly mixed by pipetting up and down five times and then immediately transferred to cells. All pipetting steps were done in a laminar flow cell culture hood and standardized to 15 min for each plate before transferring the plate of cells to a 37° C. CO₂ incubator for 1.5 h before addition of 500 μl DMEM supplemented with 10% FBS and another incubation round for 3.5 h in the 37° C. CO₂ incubator (alternatively cells were incubated 2 h with peptides and another 4 h with DMEM, 10% FBS (FIG. 8b, c, d). For the transduction time-course, cells were incubated with peptides in transduction buffer until indicated time-points. All cells were trypzinized and collected in 250 μl OptiMEM without phenol red and analyzed by FACS (GFP, FSC/SSC). 4000 viable cells were analyzed per sample. Data is presented as fold change in fluorescence compared to non-treated cells.

Cell morphology and cell viability. Cell morphology was determined by FACS analysis of FSC and SSC. Gates were set manually for viable cells using untreated control cells as reference and the fraction of viable cells compared to non-viable cells was determined for each sample. Data are presented as the relative difference compared to non-treated control cells. Viable cells per sample were determined by measuring number of viable cells that were analyzed per second by FACS. Gates for viable cells were set manually using untreated control cells as reference. Data are presented as the relative difference compared to untreated control cells.

Design of a real-time, quantitative bimolecular GFP fluorescence transduction assay. Assaying real-time cellular uptake and endosomal escape of PTDs/CPPs has remained: 1) too undefined, with no possibility to quantitatively distinguish between intracellular uptake vs. peptides stuck to the cell surface or trapped in endosomes; 2) too non-specific, with conjugation of chemical dyes that may add unwanted effects on both cells and on PTDs/CPPs leading to wrong interpretations regarding delivery; and 3) too indirect, relying on secondary enzymatic amplification events that does not allow for real-time quantification. In addition, utilization of small, membrane permeable, molecular cargo may result in excessive false-positives if extracellularly cleaved or separated from the PTD/CPP. Consequently, the absence of a real-time, live cell quantitative phenotypic transduction assay with a low to zero false positive rate has prevented the macromolecular delivery field from addressing important questions of uptake quantification, routes and dynamics of internalization, as well as how to improve the design of next-generation PTDs/CPPs.

To address these problems, the disclosure provides a self-assembling, bimolecular or split GFP fluorescence complementation system that was originally designed to tag and monitor proteins. GFP is composed of 11 β-strands that form a barrel structure allowing for peptidyl backbone cyclization and formation of a fluorescent chromophore. Removal of β-strand #11 (GFPβ11) (16 residues #215-230; RDHMVLHEYVNAAGIT; 1,826 Da) from an optimized superfolder GFP molecule resulted in a large (residues 1-214), non-fluorescent GFP fragment (GFPβ1-10). Importantly, co-incubation of GFPβ1-10 with the GFPβ11 peptide in trans efficiently reconstitutes GFP fluorescent chromophore bond and GFP fluorescence (FIG. 1a). To study PTD-mediated delivery, cell lines were generated that constitutively expressed the non-fluorescent GFPβ1-10 fragment and treated them in trans with a GFPβ11-PTD/CPP peptide to restore GFP fluorescence. This phenotypic assay offers several important advantages for monitoring transduction into cells: 1) the GFPβ11 peptide is too large to enter cells alone and requires PTD/CPP mediated delivery to enter the cytoplasm, resulting in a near zero false-positive rate from peptides stuck on the cell surface or trapped in endosomes, 2) due to the relatively small size and solubility of GFPβ11 peptide (16 amino acids) and PTD/CPP peptides (˜8-25 amino acids), GFPβ11-PTD/CPP peptides are small enough to be efficiently synthesized by a solid-state peptide synthesizer, which makes them easy to design, achieve high purification yields, and allows for a comparative transduction assay for most labs around the world, 3) the transduction process and escape into the cytoplasm can be quantitatively monitored in real-time by flow cytometry (FACS), an instrument available to most labs, and lastly 4) unlike signal amplifying indirect measuring assays, such as the TAT-Cre recombinase or splice correction assays that do not directly correlate with the number of macromolecules delivered inside of cells, transduced GFP□11-PTD/CPP peptide complementation of GFPβ1-10 induces GFP fluorescence at a 1:1 ratio that allows for a direct quantitative measurement of GFPβ11 peptides that have escaped the endosomes and are present in the cytoplasm.

Cell clones of various human cell lines constitutively expressing the non-fluorescent large GFPβ1-10 fragment, including H1299 non-small cell lung carcinoma, HaCaT immortalized keratinocytes, and MDA-MB-231 and MCF7 breast carcinomas, and assayed for reconstituted intracellular GFP fluorescence after treatment with transducible GFPβ11-PTD/CPP peptides by FACS (FIG. 1a). Starting with human H1299 cells, known to be a good model for PTD/CPP-mediated uptake, two GFPβ1-10 expressing clones were generated (c#G3 and c#G4) (FIG. 8a). GFPβ1-10 expressing H1299c#G3 cells were treated for 1.5 h with increasing amounts of transducible GFPβ11-TAT peptide (0-60 μM), followed by an additional 3.5 h in DMEM supplemented with 10% FBS. At 5 h post-addition of GFPβ11-TAT peptide, cells were trypsinized and live cell assayed by FACS. A dose-dependent GFPβ11-TAT peptide induced GFP fluorescence signal was detected (FIG. 1b, c,). Similar results were obtained with GFPβ¹⁻¹⁰ expressing H1299-c#G4 cells (FIG. 8c). GFP fluorescence correlated near linearly in both H1299 clones to increasing amounts of GFPβ11-TAT peptide addition, suggesting the absence of a critical threshold concentration for cellular uptake. Virtually all cells are transduced with GFPβ11-TAT peptide to induce GFP fluorescence, whereas the addition of control GFPβ11peptide (no TAT) plus TAT peptide in trans (non-covalent) failed to induce GFP fluorescence above background (FIG. 1c; FIG. 8b, c). GFPβ11-TAT peptide treated GFPβ1-10 H1299 cells did not display any cytotoxicity or morphological changes (FIG. 1d, e). Likewise, treatment of parental control H1299 cells (no GFPβ1-10 fragment) with GFPβ11-TAT peptide failed to increase fluorescence above background (FIG. 8d).

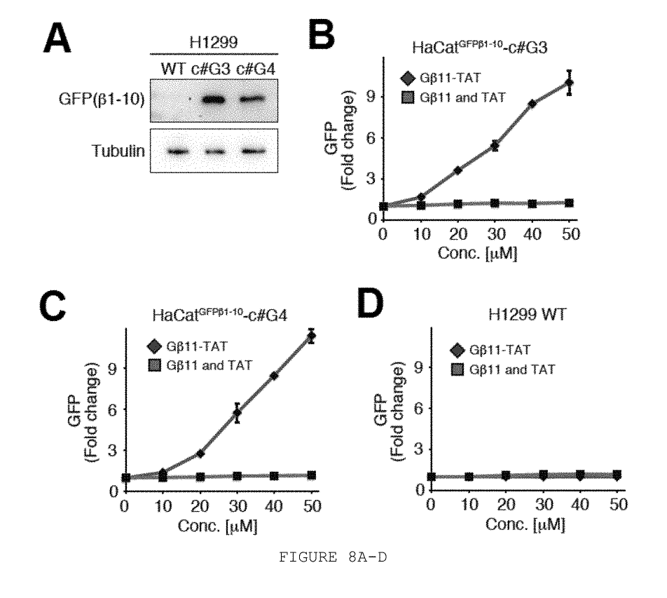

The kinetics of PTD/CPP-mediated delivery was then assayed by incubating GFPβ1-10 H1299 cells with GFPβ11-TAT peptide for various amounts of time. Complementation of GFPβ1-10 by GFPβ11peptide requires a time-dependent GFP chromophore maturation (backbone peptidyl cyclization) after binding of GFPβ¹¹ peptide to induce fluorescence. In vitro mixing of GFPβ11-TAT peptide with purified GFPβ1-10 protein fragment at 37° C. resulted in a steady time-dependent increase that started to plateau at 1 h and reached maximal GFP fluorescence by 2-4 h (FIG. 9). In GFPβ1-10 H1299 cells, GFP fluorescence was first detected 20 min after addition of GFPβ11-TAT peptides by FACS with a steady increase that peaked 9-fold over background by -2 h (FIG. 1f, g). Fluorescent video microscopy also showed a consistent increase in GFPβ11-TAT peptide treated cells throughout the entire population. The GFP signal remained relatively constant from 2 h to 10 h, but significantly decayed by 24 and 48 h post transduction, likely due to GFPβ11 peptide degradation and dilution after cell division (FIG. 1d, e). These results showed only a short delay of GFP fluorescence compared to the timing of mixed purified components in vitro, suggesting that the GFPβ11-TAT peptide rapidly (within minutes) induced macropinocytosis, escaped from the macropinosomes (endosomes) into the cytoplasm, bound to GFPβ1-10 protein fragment target and induced GFP fluorescence.

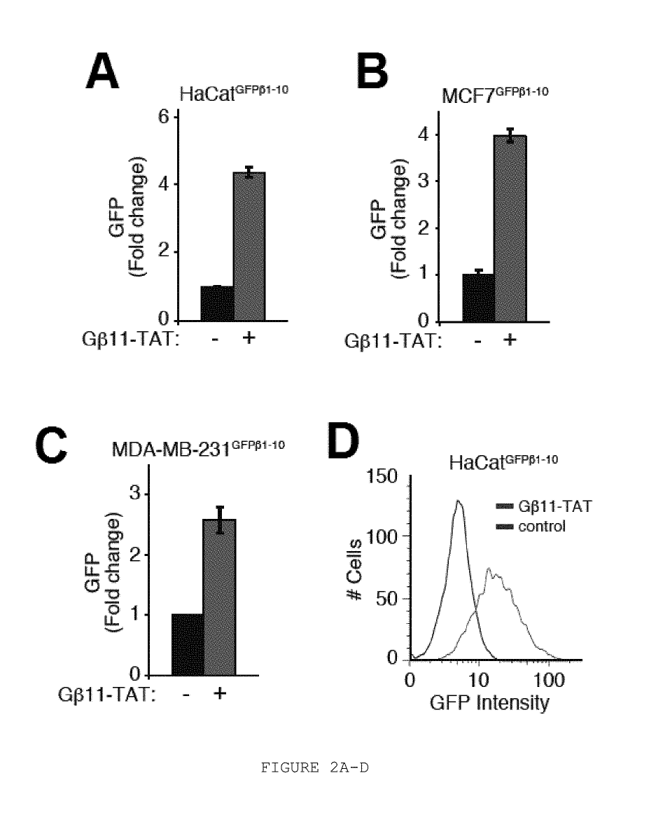

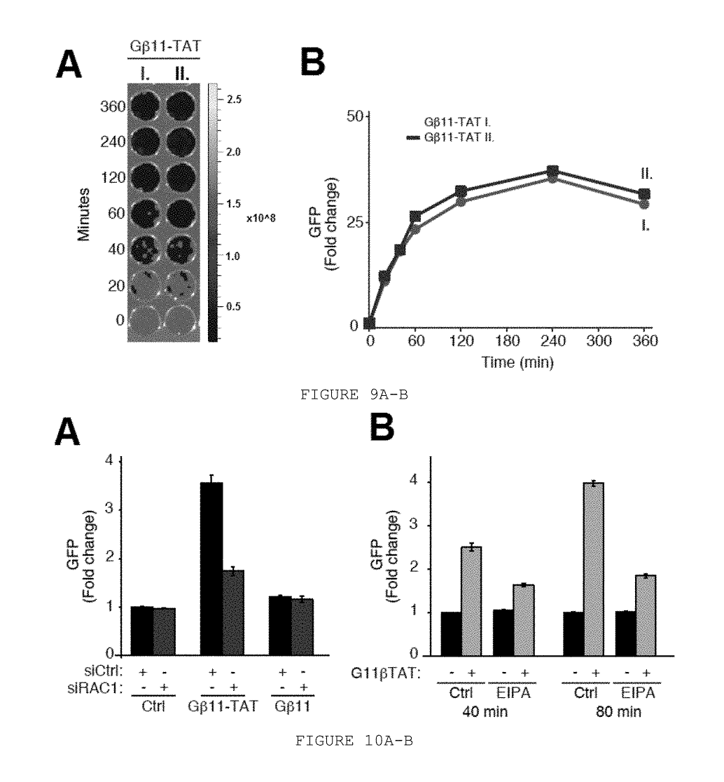

To test cell-to-cell transduction variation, three additional GFPβ1-10 protein fragment expressing cell lines were generated: MCF7c#G7, MDA-MB-231c#G3 and HaCatc#G7. Treatment of all three GFPβ1-10 cell lines with GFPβ11-TAT peptides resulted in a strong GFP fluorescence signal over background (FIG. 2a-d). Similar to GFPβ1-10 H1299 cells, FACS histogram analyses showed that most, if not all, individual cells were transduced by the GFPβ11-TAT peptide to complement GFPβ1-10 fluorescence. Consistent with an actin-dependent macropinocytosis uptake mechanism, RNAi knockdown of Rac-1 or treatment with the EIPA macropinocytosis inhibitor perturbed uptake of GFPβ11-TAT peptide resulting in a grossly decreased GFP fluorescence to near background levels (FIG. 10a, b). Taken together, the split GFP complementation assay is a rapid and robust live cell, real-time quantitative PTD/CPP uptake phenotypic assay that only measures PTD/CPP cytoplasmic delivery of GFPβ11 macromolecular peptide cargo after endosomal escape and has a near zero false positive rate.

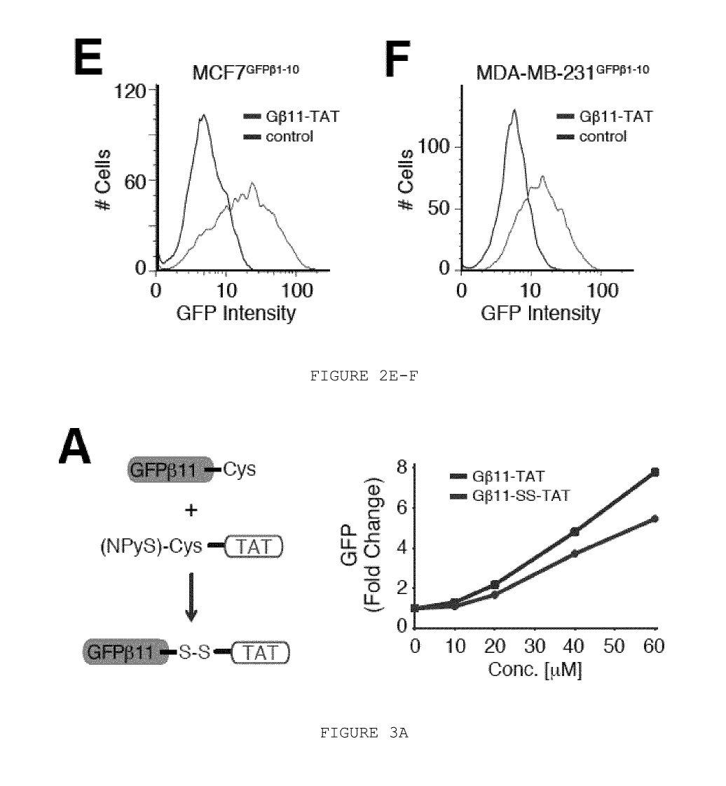

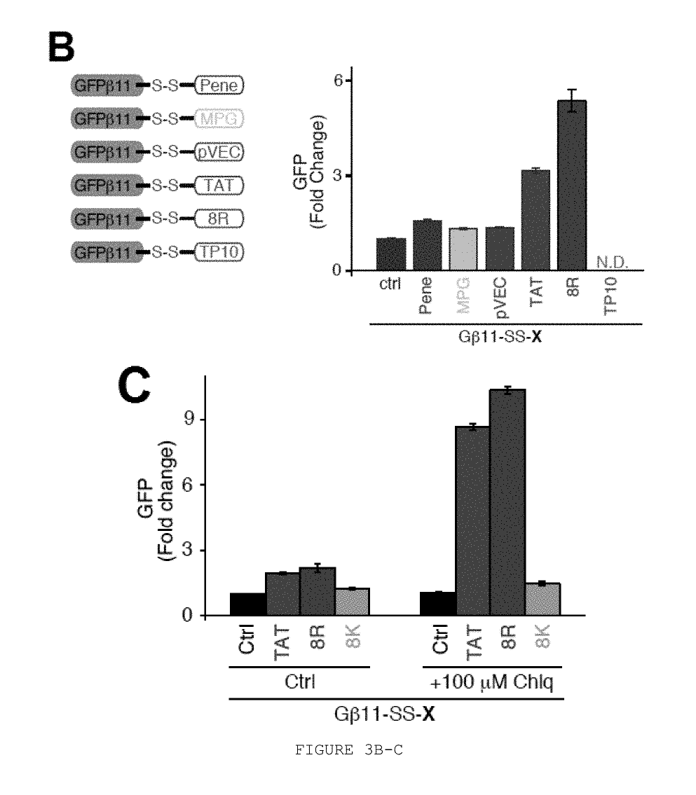

Quantitative comparison of CPP/PTD delivery peptides. Due to the structural simplicity of the GFPβ11 macromolecular peptide cargo (16 residues) combined with the direct GFP complementation readout, the split GFP transduction assay allowed for a quantitative real-time phenotypic based comparison of the intracellular delivery potential of various PTD/CPP domains. To avoid any potential interference of PTD/CPP delivery domains with GFPβ11 complementation of the cytoplasmic GFPβ1-10 protein fragment, GFPβ11 was conjugated to PTD/CPP peptides via a disulfide linker that allows for intracellular reductive separation (FIG. 3a). All disulfide conjugated peptides were purified by HPLC and quality controlled by mass-spectrometry. To evaluate any potential problems using a disulfide linker with GFP fluorescent complementation, fully synthesized GFPβ11-TAT peptide were compared to disulfide conjugated GFPβ11-(S-S)-TAT peptide for induction of GFP fluorescence. Both GFPβ11-TAT and GFPβ11-(S-S)-TAT peptide treatment of GFPβ1-10 H1299 cells induced GFP fluorescence in a dose-dependent manner (FIG. 3a). GFPβ11-(S-S)-TAT complemented GFPβ1-10 slightly lower than GFPβ11-TAT, which was attributed to unwanted reductive cleavage prior to endosomal escape into the cytoplasm. Overall, the GFPβ11-(S-S)-PTD/CPP approach allows for a direct head-to-head comparison of PTD/CPP domains regardless of size or composition.

Six of the most widely used and reported PTD/CPP delivery domains, including TAT, 8xArg, Penetratin (Antp), pVEC, MPG, and TP10 (Table 2) were conjugated to GFPβ11 peptides. All conjugates were purified by HPLC and analyzed by mass spectrometry. Treatment of GFPβ1-10 H1299 cells with 30 μM GFPβ11-(S-S)-PTD/CPP peptides resulted in various degrees of GFP fluorescence (FIG. 3b). As expected, GFPβ11-(S-S)-TAT induced a robust intracellular GFP fluorescence in the absence of cytotoxicity (FIG. 3b). Interestingly, compared to TAT, the more Arginine rich 8xArg, promoted a significantly better uptake whereas the lesser Arginine containing peptide, Antp, induced a less than two-fold increase in GFP fluorescence (FIG. 3b). In contrast, GFPβ11 peptide delivery by MPG or pVEC domains resulted in GFP complementation that was only slightly above background. TP10-mediated delivery of GFPβ11 peptide could not be determined due to a very high level of cytotoxicity (FIG. 3b).

TABLE 2
PTD/CPP peptides used in this study.*

| | |
|---|---|
| GFPβ¹¹-TAT (SEQ ID NO: 29) | RDHMVLHEYVNAAGIT-GGSGG-RKKRRQRRR |
| GFPβ¹¹-(S-S)-TAT (SEQ ID NO: 30) | RDHMVLHEYVNAAGIT-GGSGGC-(S-S)-CGG- |
| | RKKRRQRRR |
| GFPβ¹¹-(S-S)-TAT-WW (SEQ ID NO: 31) | RDHMVLHEYVNAAGIT-GGSGGC-(S-S)-CGG- |
| | RKKRRQRRR-GWWG |
| GFPβ¹¹-(S-S)-TAT-P6-GG (SEQ ID | RDHMVLHEYVNAAGIT-GGSGGC-(S-S)-CGG- |
| NO: 32) | RKKRRQRRR-(PEG)₆-GG |
| GFPβ¹¹-(S-S)-TAT-P6-WW (SEQ ID | RDHMVLHEYVNAAGIT-GGSGGC-(S-S)-CGG- |
| NO: 33) | RKKRRQRRR-(PEG)₆-GWWG |
| GFPβ¹¹-(S-S)-TAT-P12-WW (SEQ ID | RDHMVLHEYVNAAGIT-GGSGGC-(S-S)-CGG- |
| NO: 34) | RKKRRQRRR-(PEG)₁₂-GWWG |
| GFPβ¹¹-(S-S)-TAT-P18-WW (SEQ ID | RDHMVLHEYVNAAGIT-GGSGGC-(S-S)-CGG- |
| NO: 35) | RKKRRQRRR-(PEG)₁₈-GWWG |
| GFPβ¹¹-(S-S)-TAT-P6-FF (SEQ ID | RDHMVLHEYVNAAGIT-GGSGGC-(S-S)-CGG- |
| NO: 36) | RKKRRQRRR-(PEG)₆-GFFG |
| GFPβ¹¹-(S-S)-TAT-P6-W | RDHMVLHEYVNAAGIT-GGSGGC-(5-5)-CGG- |
| (SEQ ID NO: 37) | RKKRRQRRR-(PEG)₆-GWG |
| GFPβ¹¹-(S-S)-TAT-P6-FW (SEQ ID | RDHMVLHEYVNAAGIT-GGSGGC-(S-S)-CGG- |
| NO: 38) | RKKRRQRRR-(PEG)₆-GFWG |
| GFPβ¹¹-(S-S)-TAT-P6-FWF (SEQ ID | RDHMVLHEYVNAAGIT-GGSGGC-(S-S)-CGG- |
| NO: 39) | RKKRRQRRR-(PEG)₆-GFWFG |
| GFPβ¹¹-(S-S)-TAT-P6-WGGW (SEQ ID | RDHMVLHEYVNAAGIT-GGSGGC-(S-S)-CGG- |
| NO: 40) | RKKRRQRRR-(PEG)₆-GWGGWG |
| GFPβ¹¹-(S-S)-TAT-P6-WWW (SEQ ID | RDHMVLHEYVNAAGIT-GGSGGC-(S-S)-CGG- |
| NO: 41) | RKKRRQRRR-(PEG)₆-GWWWG |
| GFPβ¹¹-(S-S)-TAT-P6-FFFF (SEQ ID | RDHMVLHEYVNAAGIT-GGSGGC-(S-S)-CGG- |
| NO: 42) | RKKRRQRRR-(PEG)₆-GFFFFG |
| GFPβ¹¹-(S-S)-8xArg (SEQ ID NO: 43) | RDHMVLHEYVNAAGIT-GGSGGC-(S-S)-CGG- |
| | RRRRRRRR |
| GFPβ¹¹-(S-S)-Antp (SEQ ID NO: 44) | RDHMVLHEYVNAAGIT-GGSGGC-(S-S)-CGG- |
| | RQIKIWFQNRRMKWKK |
| GFPβ¹¹-(S-S)-MPG (SEQ ID NO: 45) | RDHMVLHEYVNAAGIT-GGSGGC-(S-S)-CGG- |
| | GALFLGFLGAAGSTMGAWSQPKKKRKV |
| GFPβ¹¹-(S-S)-TP10 (SEQ ID NO: 46) | RDHMVLHEYVNAAGIT-GGSGGC-(S-S)-CGG- |
| | AGYLLGKINLKALAALAKKIL |
| GFPβ¹¹-(S-S)-pVEC (SEQ ID NO: 47) | RDHMVLHEYVNAAGIT-GGSGGC-(S-S)-CGG- |
| | LLIILRRRIRKQAHAHSK |
| GFPβ¹¹-(S-S)-8xLys(SEQ ID NO: 48) | RDHMVLHEYVNAAGIT-GGSGGC-(S-S)-CGG- |
| | KKKKKKKK |
| GFPβ¹¹ (SEQ ID NO: 28) | RDHMVLHEYVNAAGIT |
| TAT (SEQ ID NO: 6) | RKKRRQRRR |

Arginine residues contain a bi-dentate guanidinium positive charge that is thought to form an ionic bond with cell surface bi-dentate anionic sulfates, phosphates and/or carboxylic acid groups present on sugars, lipids and proteins to stimulate macropinocytotic uptake and facilitate endosomal escape. To investigate the bi-dentate positive charge requirements for transduction, 8xArg's bi-dentate positive charges were replaced with 8xLys mono-dentate positive charges. GFPβ11-(S-S)-8xLys peptide failed to induce GFP fluorescence to any high degree, even after addition of Chloroquine, an endosomal escape enhancing drug (FIG. 3c). This quantitative head-to-head phenotypic comparison is consistent with many other reports showing a qualitative superiority of Arginine containing PTDs/CPPs by cell association of dye-labeled PTDs/CPPs.

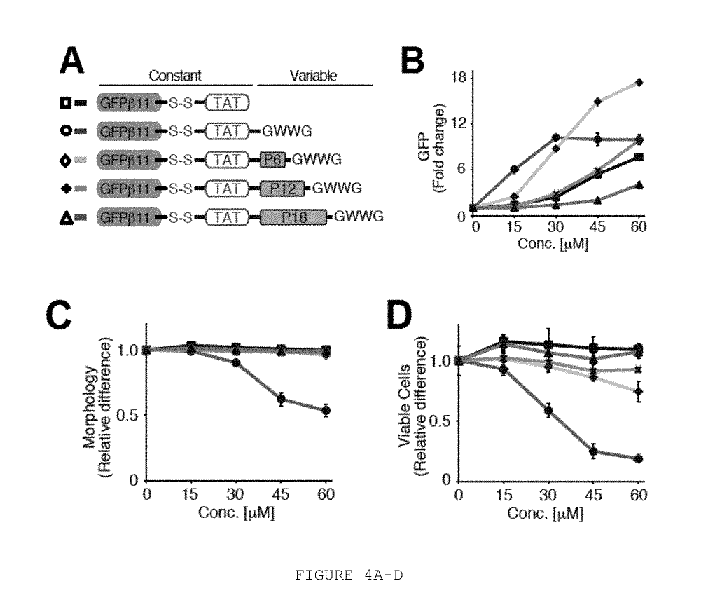

Optimizing PTD/CPP delivery by addition of hydrophobic endosomal escape domain. Successful delivery of macromolecular cargo requires that PTDs/CPPs perform two steps: 1) cell association and stimulation of endocytosis; and 2) facilitate endosomal escape. For macromolecular delivery agents, including everything from PTDs/CPPs to lipid nanoparticles, a rate-limiting step for intracellular delivery is escape from endosomes into the cytoplasm in a non-cytotoxic fashion. It has been shown that inclusion of a limited number of aromatic ring containing amino acids (Phe or Trp) resulted in enhanced dye uptake efficiency. These observations were tested using the real-time GFPβ11 macromolecular peptide phenotypic assay (FIG. 4). Addition of two Tryptophan residues flanked by Glycine residues for free bond rotation (GWWG) to the C-terminus of GFPβ11-(S-S)-TAT peptide led to a substantially higher induction of GFP fluorescence compared to GFPβ11-(S-S)-TAT alone, but it simultaneously severely increased cytotoxicity (FIG. 4c, d), thereby limiting the use of this combination. Research has shown that PTD/CPP transduction that includes a fluorescent dyes (which often contain three or four hydrophobic aromatic rings) resulted in a PTD peptide with a significantly higher degree of cytotoxicity compared to the non-dye containing PTD/CPP. Consequently, while aromatic residues enhanced endosomal escape they did so at the expense of significantly increased cytotoxicity.

Experiments were performed in an attempt to harness the use of aromatic residues to enhance endosomal escape by increasing the distance separating the PTD/CPP from the hydrophobic motif with a molecular spacer. Polyethylene glycol (PEG) is a hydrophilic, non-ionic, biologically inert polymer that is commonly used to improve the formulation and deliverability of various drugs. A GFPβ11-(S-S)-TAT-PEG(n)-GWWG delivery domain peptide was generated with an increasing number of PEG units between the TAT PTD and the hydrophobic motif, and assayed for alterations in endosomal escape by phenotypic GFP fluorescence complementation and cytotoxicity (FIG. 4). Interestingly, inclusion of a six PEG unit (P6) spacer between the TAT PTD and the GWWG motif retained enhanced cytoplasmic delivery, but completely removed cellular toxicity, even at the highest concentration tested (60 μM). However, increasing the spacer distance to 12 or 18 PEG units (P12, P18) between the TAT PTD and the GWWG motif led to a lower escape efficiency (FIG. 4a, b). Surprisingly, addition of 18 PEG units reduced GFPβ11 delivery below that of the control GFPβ11-(S-S)-TAT without any hydrophobic motif, suggesting that long PEG polymers interfere with TAT PTD/CPP-mediated uptake. Collectively, the experiments identified a hydrophobic-PEG6 Endosomal Escape Domain (EED) that combines an optimal linker length between the PTD/CPP delivery domain and the hydrophobic motif that neutralizes the cytotoxicity, while retaining the endosomal escape enhancing properties in the absence of cytotoxicity. These data reveal a new category of PTDs/CPPs that takes advantage of TAT PTD active stimulation of uptake while adding the benefits of aromatic amino acids to greatly enhance endosomal escape and cytoplasmic delivery.

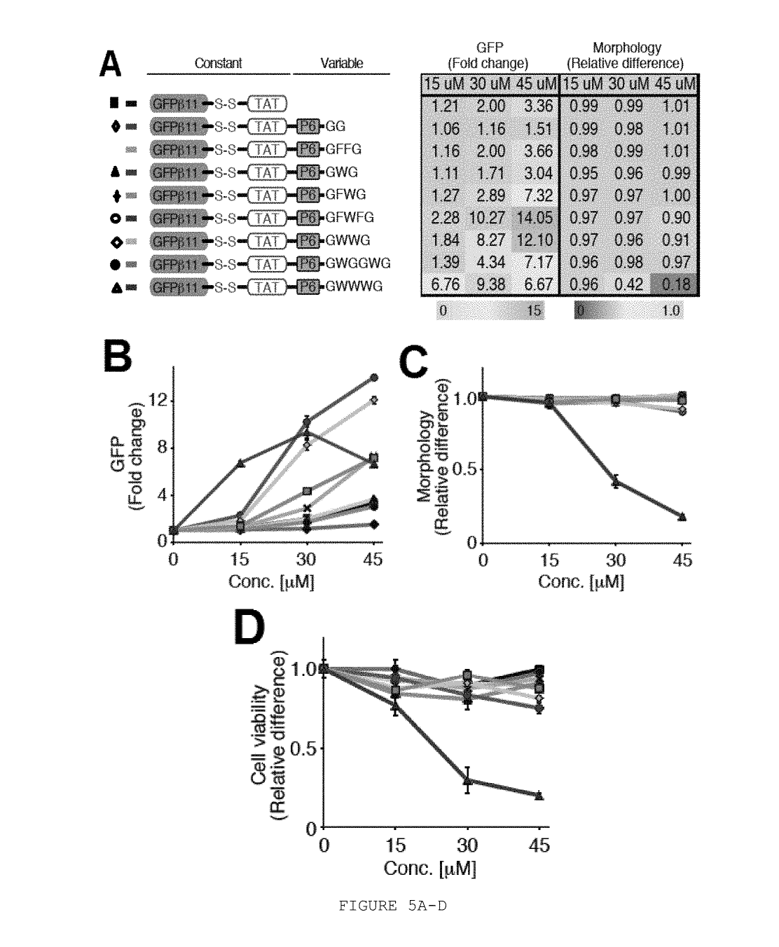

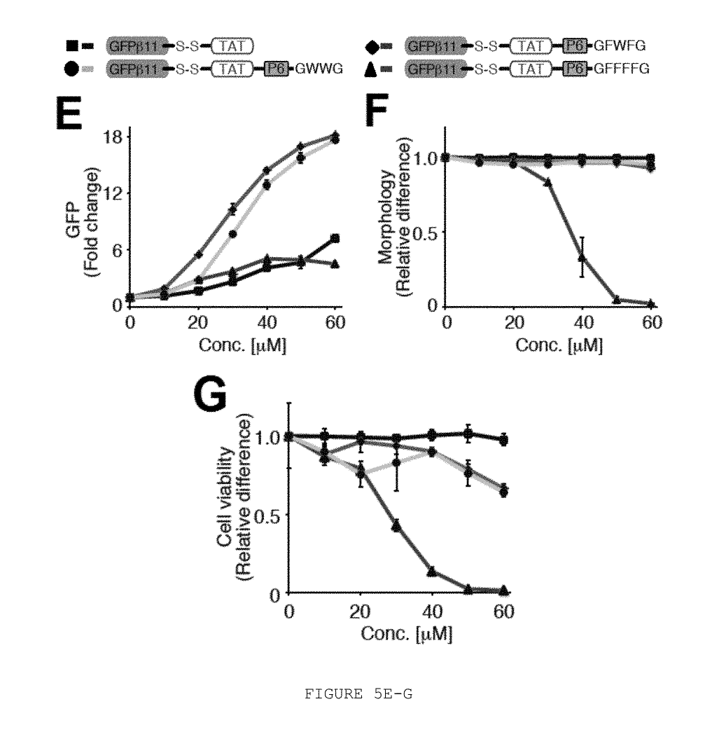

Experiments were then performed to optimize the design of EED GFPβ11-(S-S)-TAT-P6-X peptides by systematically changing the C-terminal hydrophobic X-domain with various combinations of hydrophobic residues (FIG. 5a). Both Trp (W) and Phe (F) residues are known to destabilize cellular membranes by burying their hydrophobic R groups into the lipid bilayer. To further enhance endosomal escape without negatively affecting the cell membrane, 7 different hydrophobic motif combinations were tested: -GFFG, -GWG, -GFWG, -GFWFG, -GWWG, -GWGGWG and -GWWWG plus controls for the ability to enhance GFPβ11 complementation of GFPβ1-10 fluorescence (FIG. 5a-d). The addition of two aromatic rings from either two-Phe residues (GFPβ11-(S-S)-TAT-P6-GFFG) or one Trp residue (GFPβ11-(S-S)-TAT-P6-GWG) to the C-terminus had no net effect on GFPβ11 complementation of GFPβ1-10 fluorescence compared to the parental GFPβ11-(S-S)-TAT peptide. However, the addition of hydrophobic residues containing three aromatic rings (GFPβ11-(S-S)-TAT-P6-GFWG) showed a two-fold increase in transduction compared to the parental GFPβ¹¹-(S-S)-TAT peptide with no signs of cytotoxicity (FIG. 5a-d). Moreover, addition of four C-terminal aromatic rings by inclusion of either Phe-Trp-Phe residues (GFPβ11-(S-S)-TAT-P6-GFWFG) or two Trp residues (GFPβ11-(S-S)-TAT-P6-GWWG) to the C-terminus resulted in a four-fold increase in transduction in the absence of cytotoxicity. Increasing the spacing between the two Trp residues by addition of two Gly residues -GWGGWG decreased the enhancement approximately two-fold compared to the -GWWG motif, suggesting that the enhanced endosomal escape requires a localized area of membrane destabilization. However, addition of six aromatic rings by inclusion of three Trp residues (GFPβ11-(S-S)-TAT-P6-GWWWG) resulted in a dramatic increase in cytotoxicity that hampered uptake (FIG. 5a-d). The addition of a control C-terminal PEG6-GG tail (GFPβ11-(S-S)-TAT-P6-GG) showed lower uptake than GFPβ11-(S-S)-TAT peptide, again suggesting that a free PEG polymer tail alone can interfere with TAT PTD/CPP-mediated delivery.

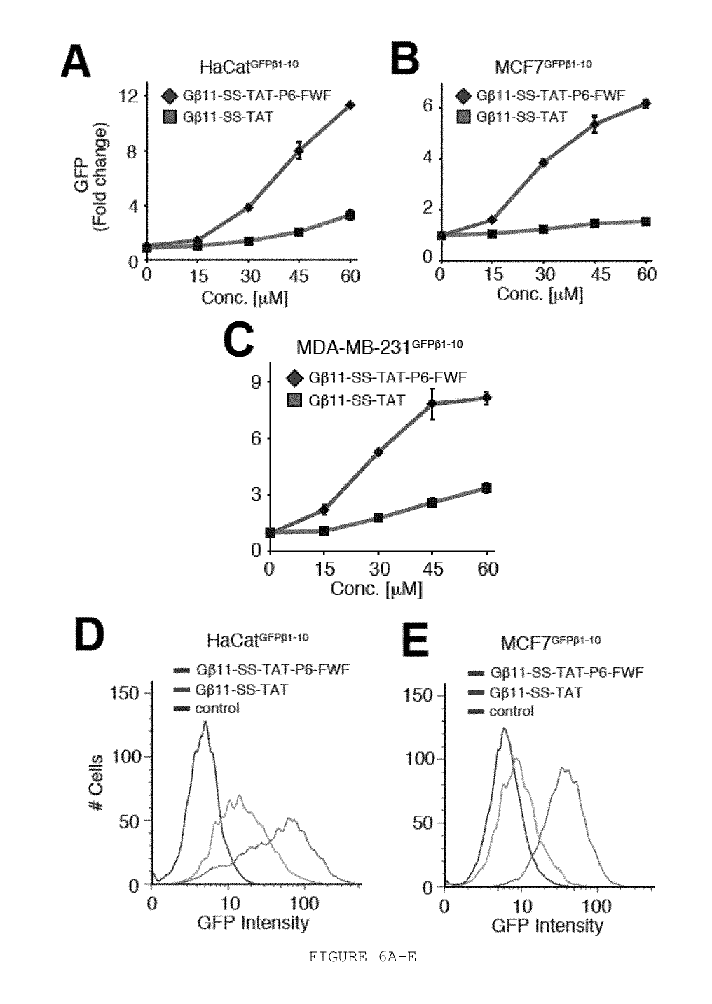

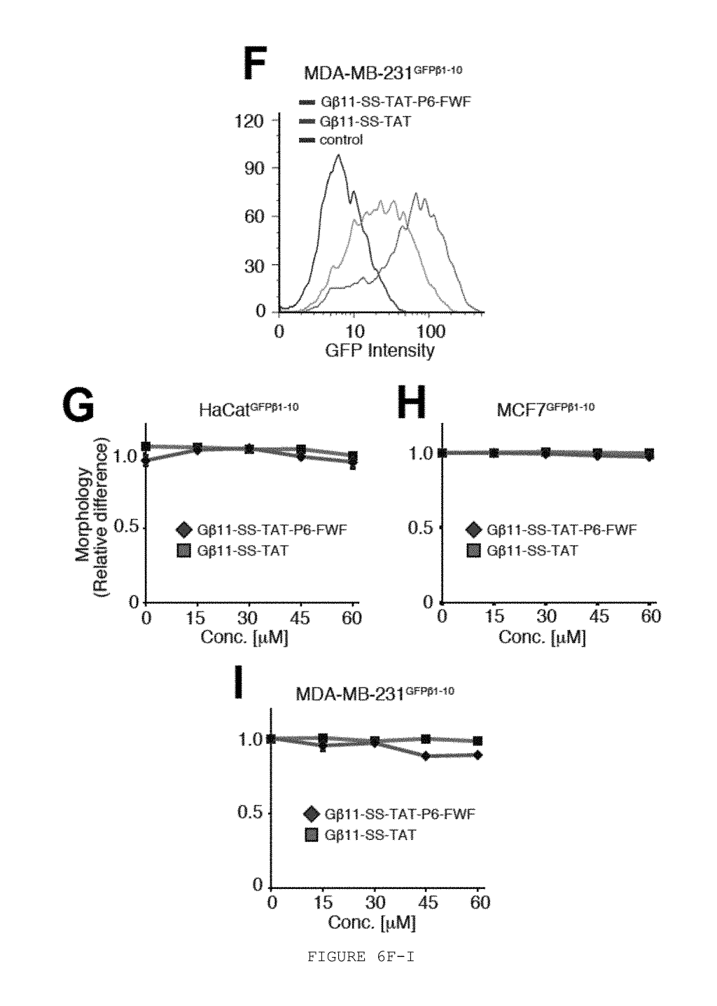

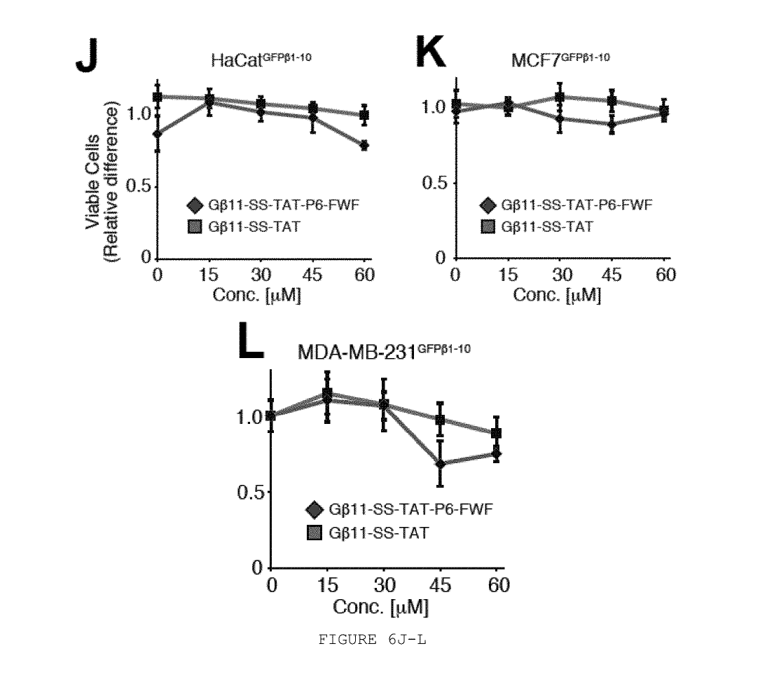

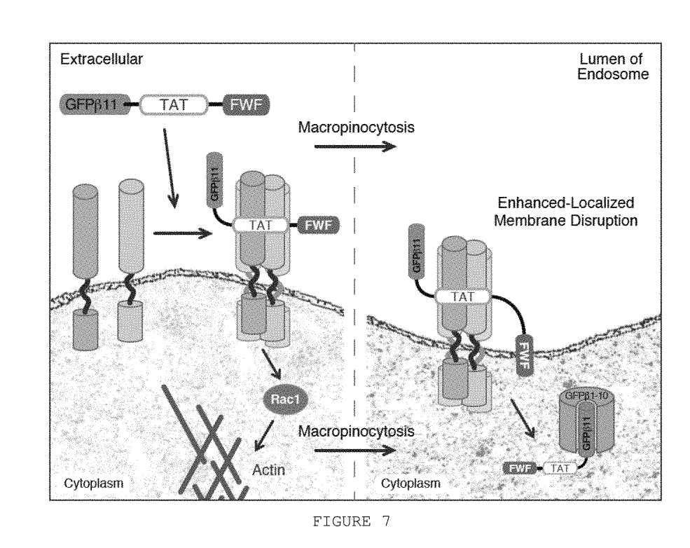

TAT-EED peptides containing four aromatic rings were optimal for both high uptake and low to no cytotoxicity, therefore increasing doses of three variants were tested: -GWWG, -GFWFG and -GFFFFG (FIG. 5e-g). Surprisingly, while both the -GWWG, and -GFWFG domains enhanced cytoplasmic escape up to 5-fold compared to parental GFPβ11-(S-S)-TAT in the absence of any detectable cytotoxicity, the four chain Phe residue -GFFFFG motif had adverse effects on cells, causing morphological changes and cell death (FIG. 5e-g), suggesting that too long of a hydrophobic patch results in cell membrane destabilization leading to cytotoxicity. Lastly, the optimized TAT-EED GFPβ11-(S-S)-TAT-P6-GFWFG peptide was compared to parental GFPβ11-(S-S)-TAT peptide in three additional GFPβ1-10 expressing cell lines, MCF7c#G7, MDA-MB-231c#G3 and HaCat-c#G7 (FIG. 6). Addition of increasing amounts of GFPβ11-(S-S)-TAT-P6-GFWFG peptide showed significantly enhanced GFP complementation in all three cell lines compared to parental GFP□11-(S-S)-TAT control peptide (FIG. 6). Low to no cytotoxicity was observed in all three treated cell lines, suggesting a universally enhanced cellular uptake and escape of PTD/CPP peptide and cargo by TAT-P6-GFWFG. Thus, using a real-time, quantitative live cell phenotypic split GFP complementation assay, the data demonstrate that the TAT-EED peptide design containing a four aromatic ring hydrophobic patch, either as -GFWFG or -GWWG, at an optimal six PEG unit distance from the TAT PTD/CPP results in a dramatically enhanced endosomal escape into the cytoplasm in a non-cytotoxic fashion.

A number of embodiments have been described. Nevertheless, it will be understood that various modifications may be made without departing from the spirit and scope of the description. Accordingly, other embodiments are within the scope of the following claims.

1. A fusion polypeptide comprising:
a) a protein transduction domain (PTD), the transduction domain comprising a membrane transport function;
b) an aromatic-rich peptide domain; and
c) a heterologous domain,
wherein the PTD is operably linked to the aromatic-rich peptide domain and the heterologous domain.

2. The fusion polypeptide of claim 1, wherein the protein transduction domain is selected from the group consisting of a polypeptide comprising a herpesviral VP22 domain; a polypeptide comprising a human immunodeficiency virus (HIV) TAT domain; a polypeptide comprising a homeodomain of an Antennapedia protein (Antp HD) domain; an N-terminal cationic prion protein domain; and functional fragments thereof.

3. The fusion polypeptide of claim 1, wherein the protein transduction domain comprises a sequence selected from the group consisting of SEQ ID NO:7 from amino acid 47-57; B1-X₁-X₂-X₃-B₂-X₄-X₅-B_{3,} wherein B_{1,} B_{2,} and B₃ are each independently a basic amino acid, the same or different and X_{1,} X_{2,} X_{3,} X₄ and X₅ are each independently an alpha-helix enhancing amino acid the same or different (SEQ ID NO:1); B1-X₁-X₂-B₂-B₃-X₃-X₄-B_{4,} wherein B_{1,} B_{2,} B_{3,} and B₄ are each independently a basic amino acid, the same or different and X_{1,} X_{2,} X_{3,} and X₄ are each independently an alpha-helix enhancing amino acid the same or different (SEQ ID NO:2); X-X-R-X-(P/X)-(B/X)-B-(P/X)-X-B-(B/X), wherein X is any alpha helical promoting residue such as alanine; P/X is either proline or X as previously defined, B is a basic amino acid residue and B/X is either B or X as defined above (SEQ ID NO:4); a sequence of about 7 to 10 amino acids and containing KX₁RX₂X_{1,} wherein X₁ is R or K and X₂ is any amino acid (SEQ ID NO:5); RKKRRQRRR (SEQ ID NO:6); and KKRPKPG (SEQ ID NO:3).

4. The fusion polypeptide of claim 1, wherein the heterologous domain comprises a diagnostic and/or therapeutic agent.

5-22. (canceled)

23. The fusion polypeptide of claim 1, wherein the aromatic-rich peptide domain comprises 1 to 8 amino acids and comprises from 3-5 aromatic rings.

24. The fusion polypeptide of claim 1, wherein the aromatic rich peptide domain further comprises a hydrophilic polymer spacer between the PTD and the aromatic-rich peptide domain.

25. The fusion polypeptide of claim 24, wherein the hydrophilic polymer spacer comprises polyethylene glycol (PEG) having 1-18 PEG moieties.

26. The fusion polypeptide of claim 1, wherein the fusion polypeptide comprises the aromatic-rich peptide domain and a PEG linker (endosomal escape domain (EED)).

27. The fusion polypeptide of claim 26, wherein the endosomal escape domain (EED) comprises of 1 to 8 amino acids comprising from 3-5 aromatic groups and a spacer of 2-18 PEG moieties.

28. The fusion polypeptide of claim 26, wherein the EED comprises 4 aromatic groups.

29. The fusion polypeptide of claim 28, wherein the EED does not comprise more than 3 phenylalanines in series.

30. The fusion polypeptide of claim 1 wherein the aromatic-rich peptide domain comprises a peptide selected from the group consisting of GFFG, GWG, GFWG, GFWFG, GWWG and GWGGWG.

31. The fusion polypeptide of claim 1, having the general formula: Z-PTD-(PEG)ₓ-(aromatic amino acids)₂₋₄ wherein x is 2-18 and Z is the heterologous domain.

32. The fusion polypeptide of claim 31 having the general formula selected from the group consisting of: Z-PTD-(PEG)ₓ-GFFG, Z-PTD-(PEG)ₓ-GWG, Z-PTD-(PEG)ₓ-GFWG, Z-PTD-(PEG)ₓ-GFWFG, Z-PTD-(PEG)ₓ-GWWG and Z-PTD-(PEG)ₓ-GWGGWG.

33. A pharmaceutical composition comprising the fusion polypeptide of claim 1.

34. A method of introducing a therapeutic and/or diagnostic agent in to a target cell, the method comprising contacting the cell with the fusion polypeptide of claim 1.

35. The method of claim 34, wherein the contacting is in vivo or in vitro.

36-38. (canceled)

39. A method of identifying a cell comprising a phenotype of interest in a subject, the method comprising contacting the subject with a fusion polypeptide of claim 1, wherein the heterologous domain comprises a diagnostic agent.

40. A fusion polypeptide comprising:
a) a protein transduction domain (PTD), the transduction domain comprising a membrane transport function; and
b) a peptide comprising SEQ ID NO:28.

41-43. (canceled)

44. The fusion polypeptide of claim 40, further comprising an endosomal escape domain and/or a targeting ligand domain.

45. (canceled)

46. A method of measuring transport of a molecule into a cell comprising contacting a cell comprising the N-terminal domain of green fluorescent protein comprising a sequence that is at least 90% identical to SEQ ID NO:27 from amino acid 1-214, with a fusion polypeptide of claim 40 and measuring fluorescence.

47. An isolated polynucleotide encoding the fusion polypeptide of claim 1.

48. A vector comprising the polynucleotide of claim 47.

49. (canceled)

50. A host cell containing the polynucleotide of claim 47.

51. An assay system comprising a simple real-time, quantitative live cell phenotypic PTD/CPP transduction assay using a split GFP peptide cargo complementation approach that allows for a direct measurement of the transduced cargo in the cytoplasm.


SEQUENCE LISTING (48 sequences)

SEQ ID NO: 1 - protein, 8 aa, Artificial Sequence
  Peptide consensus sequence
  misc (1)..(1): X at position 1 is a basic amino acid
  misc (2)..(4): X is any alpha-helix enhancing amino acid
  misc (5)..(5): X at position 5 is a basic amino acid
  misc (6)..(7): X is any alpha-helix enhancing amino acid
  misc (8)..(8): X at position 8 is a basic amino acid
XXXXXXXX

SEQ ID NO: 2 - protein, 8 aa, Artificial Sequence
  Peptide consensus sequence
  misc (1)..(1): X at position 1 is a basic amino acid
  misc (2)..(3): X is any alpha-helix enhancing amino acid
  misc (4)..(5): X at position 4 and 5 are each independently a basic amino acid
  misc (6)..(7): X is any alpha-helix enhancing amino acid
  misc (8)..(8): X is a basic amino acid
XXXXXXXX

SEQ ID NO: 3 - protein, 7 aa, Artificial Sequence
  Prion protein fragment
KKRPKPG

SEQ ID NO: 4 - protein, 11 aa, Artificial Sequence
  Peptide consensus sequence
  misc (1)..(2): X is any alpha-helix enhancing amino acid
  misc (4)..(4): X is any alpha-helix enhancing amino acid
  misc (5)..(5): X is any alpha-helix enhancing amino acid or a proline
  misc (6)..(7): X is any alpha-helix enhancing amino acid or a basic amino acid
  misc (8)..(8): X is any alpha-helix enhancing amino acid or a proline
  misc (9)..(9): X is any alpha-helix enhancing amino acid
  misc (10)..(11): X is any alpha-helix enhancing amino acid or a basic amino acid
XXRXXXXXXXX

SEQ ID NO: 5 - protein, 5 aa, Artificial Sequence
  Peptide consensus sequence
  misc (2)..(2): X is arginine or lysine
  misc (4)..(4): X is any amino acid
  misc (5)..(5): X is an arginine or lysine
KXRXX

SEQ ID NO: 6 - protein, 9 aa, Artificial Sequence
  Synthesized cationic peptide sequence
RKKRRQRRR

SEQ ID NO: 7 - protein, 86 aa, Human immunodeficiency virus type 1
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 8 - protein, 5 aa, Artificial Sequence
  Peptide Linker Sequence
GGGGS

SEQ ID NO: 9 - protein, 5 aa, Artificial Sequence
  Peptide Linker Sequence
  misc (1)..(5): GGGGS is repeated two or more times
GGGGS

SEQ ID NO: 10 - protein, 12 aa, Artificial Sequence
  Peptide Linker Sequence
GKSSGSGSESKS

SEQ ID NO: 11 - protein, 14 aa, Artificial Sequence
  Peptide Linker Sequence
GSTSGSGKSSEGKG

SEQ ID NO: 12 - protein, 18 aa, Artificial Sequence
  Peptide Linker Sequence
GSTSGSGKSSEGSGSTKG

SEQ ID NO: 13 - protein, 18 aa, Artificial Sequence
  Peptide Linker Sequence
GSTSGSGKPGSGEGSTKG

SEQ ID NO: 14 - protein, 14 aa, Artificial Sequence
  Peptide Linker Sequence
EGKSSGSGSESKEF

SEQ ID NO: 15 - protein, 20 aa, Artificial Sequence
  HA2 analog
GLFGAIAGFIEGGWTGMIDG

SEQ ID NO: 16 - protein, 20 aa, Artificial Sequence
  HA2 analog
GLFEAIAEFIEGGWEGLIEG

SEQ ID NO: 17 - protein, 10 aa, Artificial Sequence
  Fragment DV3 peptide ligand
LGASWHRPDK

SEQ ID NO: 18 - protein, 42 aa, Artificial Sequence
  Fusion Polypeptide
LGASWHRPDKGRRRQRRKRGKKHRSTSQGKKSKLHSSHARSG

SEQ ID NO: 19 - protein, 42 aa, Artificial Sequence
  Fusion Polypeptide
LGASWHRPDKGRRRQRRKRGKKHRSTSQGEASELHSSHARSG

SEQ ID NO: 20 - protein, 11 aa, Artificial Sequence
  Fragment DV3 peptide ligand
LGASWHRPDKG

SEQ ID NO: 21 - protein, 32 aa, Artificial Sequence
  Fusion Polypeptide
RRRQRRKKRGKKHRSTSQGKKSKLHSSHARSG

SEQ ID NO: 22 - protein, 21 aa, Artificial Sequence
  Fusion Polypeptide
LGASWHRPDKGRRRQRRKKRG

SEQ ID NO: 23 - protein, 22 aa, Artificial Sequence
  Fusion Polypeptide Fragment
KKHRSTSQGKKSKLHSSHARSG

SEQ ID NO: 24 - protein, 33 aa, Artificial Sequence
  Fusion Polypeptide
LGASWHRPDKGKKHRSTSQGKKSKLHSSHARSG

SEQ ID NO: 25 - protein, 18 aa, Artificial Sequence
  Fusion Polypeptide
RKKRRQRRRGPVKRRLFG

SEQ ID NO: 26 - protein, 29 aa, Artificial Sequence
  Fusion Polypeptide
LGASWHRPDKGRKKRRQRRRGPVKRRLFG

SEQ ID NO: 27 - protein, 238 aa, Artificial Sequence
  Green Fluorescent Protein
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 28 - protein, 16 aa, Artificial Sequence
  GFP-beta-11 peptide
RDHMVLHEYVNAAGIT

SEQ ID NO: 29 - protein, 30 aa, Artificial Sequence
  GFP beta 11-TAT
RDHMVLHEYVNAAGITGGSGGRKKRRQRRR

SEQ ID NO: 30 - protein, 36 aa, Artificial Sequence
  GFP-beta-11-SS-Tat
RDHMVLHEYVNAAGITGGSGGCSSCGGRKKRRQRRR

SEQ ID NO: 31 - protein, 40 aa, Artificial Sequence
  GFP-beta-11-SS-Tat-WW
RDHMVLHEYVNAAGITGGSGGCSSCGGRKKRRQRRRGWWG

SEQ ID NO: 32 - protein, 38 aa, Artificial Sequence
  GFP-beta-11-(S-S)-TAT-P6-GG
  MISC_FEATURE (36)..(37): Residues 36 and 37 are separated by 6 Pegylation moieties (PEGs)
RDHMVLHEYVNAAGITGGSGGCSSCGGRKKRRQRRRGG

SEQ ID NO: 33 - protein, 40 aa, Artificial Sequence; identical to SEQ ID NO: 31
  GFP-beta-11-(S-S)-TAT-P6-WW
  MISC_FEATURE (36)..(37): Residues 36 and 37 are separated by 6 Pegylation moieties (PEGs)

SEQ ID NO: 34 - protein, 40 aa, Artificial Sequence; identical to SEQ ID NO: 31
  GFP-beta-11-(S-S)-TAT-P12-WW
  MISC_FEATURE (36)..(37): Residues 36 and 37 are separated by 12 Pegylation moieties (PEGs)

SEQ ID NO: 35 - protein, 40 aa, Artificial Sequence; identical to SEQ ID NO: 31
  GFP-beta-11-(S-S)-TAT-P18-WW
  MISC_FEATURE (36)..(37): Residues 36 and 37 are separated by 18 Pegylation moieties (PEGs)

SEQ ID NO: 36 - protein, 40 aa, Artificial Sequence
  GFP-beta-11-(S-S)-TAT-P6-FF
  MISC_FEATURE (36)..(37): Residues 36 and 37 are separated by 6 Pegylation moieties (PEGs)
RDHMVLHEYVNAAGITGGSGGCSSCGGRKKRRQRRRGFFG

SEQ ID NO: 37 - protein, 39 aa, Artificial sequence
  GFP-beta-11-(S-S)-TAT-P6-W
  MISC_FEATURE (36)..(37): Residues 36 and 37 are separated by 6 Pegylation moieties (PEGs)
RDHMVLHEYVNAAGITGGSGGCSSCGGRKKRRQRRRGWG

SEQ ID NO: 38 - protein, 40 aa, Artificial Sequence
  GFP-beta-11-(S-S)-TAT-P6-FW
  MISC_FEATURE (36)..(37): Residues 36 and 37 are separated by 6 Pegylation moieties (PEGs)
RDHMVLHEYVNAAGITGGSGGCSSCGGRKKRRQRRRGFWG

SEQ ID NO: 39 - protein, 41 aa, Artificial Sequence
  GFP-beta-11-(S-S)-TAT-P6-FWF
  MISC_FEATURE (36)..(37): Residues 36 and 37 are separated by 6 Pegylation moieties (PEGs)
RDHMVLHEYVNAAGITGGSGGCSSCGGRKKRRQRRRGFWFG

SEQ ID NO: 40 - protein, 42 aa, Artificial Sequence
  GFP-beta-11-(S-S)-TAT-P6-WGGW
  MISC_FEATURE (36)..(37): Residues 36 and 37 are separated by 6 Pegylation moieties (PEGs)
RDHMVLHEYVNAAGITGGSGGCSSCGGRKKRRQRRRGWGGWG

SEQ ID NO: 41 - protein, 41 aa, Artificial Sequence
  GFP-beta-11-(S-S)-TAT-P6-WWW
  MISC_FEATURE (36)..(37): Residues 36 and 37 are separated by 6 Pegylation moieties (PEGs)
RDHMVLHEYVNAAGITGGSGGCSSCGGRKKRRQRRRGWWWG

SEQ ID NO: 42 - protein, 42 aa, Artificial Sequence
  GFP-beta-11-(S-S)-TAT-P6-FFFF
  MISC_FEATURE (36)..(37): Residues 36 and 37 are separated by 6 Pegylation moieties (PEGs)
RDHMVLHEYVNAAGITGGSGGCSSCGGRKKRRQRRRGFFFFG

SEQ ID NO: 43 - protein, 35 aa, Artificial Sequence
  GFP-beta-11-(S-S)-8xArg
RDHMVLHEYVNAAGITGGSGGCSSCGGRRRRRRRR

SEQ ID NO: 44 - protein, 43 aa, Artificial Sequence
  GFP-beta-11-(S-S)-Antp
RDHMVLHEYVNAAGITGGSGGCSSCGGRQIKIWFQNRRMKWKK

SEQ ID NO: 45 - protein, 54 aa, Artificial Sequence
  GFP-beta-11-(S-S)-MPG
RDHMVLHEYVNAAGITGGSGGCSSCGGGALFLGFLGAAGSTMGAWSQPKKKRKV

SEQ ID NO: 46 - protein, 48 aa, Artificial Sequence
  GFP-beta-11-(S-S)-TP10
RDHMVLHEYVNAAGITGGSGGCSSCGGAGYLLGKINLKALAALAKKIL

SEQ ID NO: 47 - protein, 45 aa, Artificial Sequence
  GFP-beta-11-(S-S)-pVEC
RDHMVLHEYVNAAGITGGSGGCSSCGGLLIILRRRIRKQAHAHSK

SEQ ID NO: 48 - protein, 35 aa, Artificial Sequence
  GFP-beta-11-(S-S)-8xLys
RDHMVLHEYVNAAGITGGSGGCSSCGGKKKKKKKK